(19) United States
(12) Patent Application Publication
(10) Pub. No.: US 2016/0082141 A1
(43) Pub. Date: Mar. 24, 2016

(54) CHITIN AND ALGINATE COMPOSITE FIBERS

(71) Applicant: THE BOARD OF TRUSTEES OF THE UNIVERSITY OF ALABAMA, Alabama, AL (US)

(72) Inventors: Robin D. Rogers, Tuscaloosa, AL (US); Gabriella Gurau, Tuscaloosa, AL (US); Julia L. Shamshina, Tuscaloosa, AL (US); Daniel T. Daly, Tuscaloosa, AL (US)

(21) Appl. No.: 14/785,230

(22) PCT Filed: Apr. 21, 2014
(86) PCT No.: PCT/US14/34793

§ 371 Date: Oct. 16, 2015

Related U.S. Application Data
(60) Provisional application No. 61/813,892, filed on Apr. 19, 2013.

(51) Int. Cl. A61L 15/44; C08L 5/04; A61L 15/22; C08L 5/08

(52) U.S. Cl. CPC A61L 15/44; C08L 5/08; C08L 5/04; A61L 15/225; A61L 2300/428

(57) ABSTRACT

Disclosed herein are composite fibers that comprises chitin and alginate. The formation of the chitinous-alginate composite fiber involves the use of ionic liquids and high molecular weight pure chitin obtained directly from chitin biomass. Optional additive such as vitamin E is successfully incorporated in to the composite fiber. The chitinousalginate fiber formed has a continuous and homogenous morphology, even with the addition of additive. Methods of making and using the chitinous-alginate composite fiber as wound dressing is also disclosed.

### CROSS REFERENCE TO RELATED APPLICATIONS

This application claims the benefit of priority to U.S. Provisional Application No. 61/813,892, filed Apr. 19, 2013, which is incorporated by reference herein in its entirety.

### FIELD

This disclosure generally relates to chitinous-alginate composite materials and their uses in wound healing applications. The method of forming the composite material is also disclosed herein.

### BACKGROUND

Chitin, a linear amino polysaccharide composed of β-(1→4)-linked 2-acetamido-2-deoxy-β-D-glucose units found in the outer skeleton of arthropods, is the second most plentiful natural polymer after cellulose (Bartlett et al., Science, 310: 1775-1777 (2005)). Its bioactivity, biocompatibility, and low toxicity make it suitable for controlled drug release formulations, cosmetics, food preservation, fertilizers, or biodegradable packaging materials, while its ability to absorb both metal ions and hydrophobic organic compounds make it useful in waste water processing and other industrial applications (Synowiecki et al., Crit. Rev. Food Sci. Nutr., 43:145-171 (2003) and Kumar, React. Funct. Polym., 46:1-27 (2000)). However, due to its high density of hydrogen bonds, chitin is completely insoluble in water, most organic solvents, dilute acidic solutions, and dilute basic solutions. Thus, various chemical modifications have been applied to make chitin more easily soluble, including N-deacetylation to form chitosan (Sashiwa et al., Carbohydr. Polym., 39:127-138 (1999)).

Chitin can be obtained commercially in pure grade or practical grade (PG-chitin). PG-chitin is primarily produced from crustacean shells by a chemical method that involves acid demineralization of the shell, followed by removal of shell proteins by alkali treatment, and then decolorization (Percot et al., Biomacromolecules, 4:12-18 (2003)). It can be further purified by methanesulfonic acid treatment to obtain pure chitin (Hirano and Nagao, Agric. Biol. Chem., 52:2111-2112 (1988)). Crustacean shells (e.g., shrimp shells) contain not only chitin, but also large amounts of protein, mineral salts, and a small amount of lipids. Thus, crustacean shells are even harder to dissolve than either PG-chitin or pure (native) chitin. High molecular weight chitin was successfully directly extracted from biomass such as crustacean shells as reported by Qin, Rogers, and Daly, by using various ILs (see WO2010/141470, which is incorporated by reference herein in its entirety for its teaching of chitin dissolution, regeneration, and processing using ILs). The authors have subsequently further developed less chemical- and energy efficient methods and processes to extract chitin and to form chitin fibers of different thickness (see U.S. application Ser. Nos. 13/375,245; 13/505,323; and 13/428,786 and U.S. Provisional Application Nos. 61/674,979 and 61/764,770, all five of which are incorporated by reference herein in their entireties for their teaching of chitin extraction and chitin fiber generation and processing).

Chitin is biocompatible and is believed to have anti-microbial activities. The use of chitin in medical related applications however has been largely untapped due to its inert nature. For example, forming composite fiber using chitin as one of the key elements has largely been unexplored. Besides being biocompatible and bio-degradable, alginate fibers are believed to have wound healing properties and have been widely used in wound management applications including wound healing. ALGISITE M™ wound dressing for example, has been made from calcium salt of alginic acid and can be applied to exuding lesions. ALGISITE M™ dressing, however, has been shown to be inadequate for deeper wounds. CURASORB™ is another alginate product available in multiple sizes and is known for its ability to absorb liquid 20 times of it weight. Alginate fibers however can be brittle. While alginate has low strength and chitin is perceived to be inert, the seemingly incompatible properties of the two materials such as the large differences in the solubility of chitin and alginic acid polymers have rendered biocompatible materials that have both major alginate and chitin components largely unexplored. Thus, what are still needed are composite material that uses chitinous polymer and alginate as the key components and methods for forming the composite materials to further utilize and explore the special properties of such materials. The subject matter disclosed herein addresses these and other needs.

### SUMMARY

In accordance with the purposes of the disclosed materials, fibers, compositions, articles, and methods, as embodied and broadly described herein, the disclosed subject matter, in one example, relates to chitinous-alginate composite materials such as fibers and methods for preparing and using such materials. In a further aspect, disclosed herein are wound dressings using the chitinous-alginate composite materials. In still a further aspect, disclosed herein are methods of forming and using chitinous-alginate composite materials for wound healing. Also, disclosed herein are methods for forming the chitinous-alginate composite materials.

Additional advantages of the disclose subject matter will be set forth in part in the description that follows, and in part will be obvious from the description, or can be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects described below.

FIG. 1 is a schematic diagram illustrating an example chitin fiber forming process.

FIG. 2 shows two sets of examples of chitin fiber (CH); chitinous-alginate fiber (CHA); and chitinous-alginate with 10% vitamin E fiber (CHAE10).

FIG. 3A is an IR spectra of CH, CHA, CHAE10 fibers, ionic liquid, and vitamin E.

FIG. 3B is an expanded view of a portion of the IR spectra of FIG. 3A showing vitamin E related peaks.

FIG. 4 shows thermal gravimetric analysis (TGA) of CH, CHA, and CHAE10.

FIGS. 5A-5L show results from optical microscopy analysis of CH, CHA or CHAE fibers.

FIG. 6 shows SEM photographs of the surfaces of the CH, CHA or CHAE fibers.

FIG. 7 shows TEM photographs of the surfaces of the CH, CHA or CHAE fibers.

FIG. 8 shows comparison of the diameters of the CH, CHA or CHAE fibers.

FIG. 9 is an example of a wound patch using the CH, CHA or CHAE fibers.

FIG. 10 shows the rate of wounding healing using chitin fiber based dressing of FIG. 3 compared to commercial OPSITE™.

FIG. 11 shows the wound closure results expressed as percentage of initial mean wound area (averaged per group).

FIG. 12 shows representative images of the wound sites taken on days 3, 7, 10, and 14.

FIG. 13 shows histological micrographs of wound sites at day 7.

### DETAILED DESCRIPTION

The materials, compounds, compositions, and methods described herein may be understood more readily by reference to the following detailed description of specific aspects of the disclosed subject matter, the Figures, and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### GENERAL DEFINITIONS

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “a polymer” includes mixtures of two or more such polymers, reference to “the component” includes mixtures of two or more such component, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. “Heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is included in the term “aryl,” defines a group that contains an aromatic group that does not contain a heteroatom. The aryl and heteroaryl groups can be substituted or unsubstituted. The aryl and heteroaryl groups can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O⁻. An acetate or (OAc) is CH₃C(O)O⁻. Throughout the specification C(O) is used as an abbreviation for a carbonyl group.

The term “halide” as used herein refers to the halogens fluorine, chlorine, bromine, and iodine.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture.

The term “ion pair” is a positive ion (i.e., cation) and a negative ion (i.e., anion) that are temporarily bonded together by an attractive force (i.e., electrostatic, van-der-Waals, ionic).

The term “ionic liquid” describes a salt with a melting point below 150° C., whose melt is composed of discrete ions.

The term “hydrogen bond” describes an attractive interaction between a hydrogen atom from a molecule or molecular fragment X—H in which X is more electronegative than H, and an atom or a group of atoms in the same or different molecule, in which there is evidence of bond formation. The hydrogen bond donor can be a cation and the hydrogen bond acceptor can be an anion.

The term “co-crystal” describes a crystalline structure made up of two or more atoms, ions, or molecules that exist in a definite stoichiometric ratio. Generally, a co-crystal is comprised of two or more components that are not covalently bonded and instead are bonded via van-der-Waals interactions, ionic interactions or via hydrogen bonding.

The term “complex” describes a coordination complex, which is a structure comprised of a central atom or molecule that is weakly connected to one or more surrounding atoms or molecules, or describes chelate complex, which is a coordination complex with more than one bond.

The term “eutectic” is a mixture of two or more ionic liquids, ionic liquids and neutral compounds, ionic liquids and charge compounds, ionic liquids and complexes, ionic liquids and ion pairs, or two or more ion pairs that have at least one component in common.

The term ion-containing liquid is used herein to collectively refer to either an ion pair, co-crystal, or eutectic.

Reference will now be made in detail to specific aspects of the disclosed materials, compounds, compositions, articles, and methods, examples of which are illustrated in the accompanying Examples.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Also, disclosed herein are materials, compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a composition is disclosed and a number of modifications that can be made to a number of components of the composition are discussed, each and every combination and permutation that are possible are specifically contemplated unless specifically indicated to the contrary. Thus, if a class of components A, B, and C are disclosed as well as a class of components D, E, and F and an example of a composition A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific aspect or combination of aspects of the disclosed methods, and that each such combination is specifically contemplated and should be considered disclosed.

### Materials and Methods

Disclosed herein, in one aspect, are composite materials comprising chitinous-alginate as major component with optional additive. High molecular weight chitin (HW chitin) has been obtained via IL extraction technology previously disclosed (see WO2010/141470, U.S. application Ser. Nos. 13/375,245; 13/505,323; and 13/428,786 and U.S. Provisional Application Nos. 61/674,979 and 61/764,770, all six of which are incorporated by reference herein in their entirety for their teaching of chitin extraction and chitin fiber dissolution, regeneration, and processing using ILs). Commercially available chitin, chitosan, HW chitin, high molecular weight chitosan derived from HW chitin, or a combination thereof are used as chitinous source material to form the chitinous-alginate composite material with the optional additive described herein. The chitinous source is dissolved in ionic liquids or other ion-containing liquids to form a chitinous IL solution. The dissolution of chitinous source material in some embodiments is facilitated by microwave irradiation. Alginate is then added to the chitinous IL solution followed by optional addition of an additive such as vitamin E to form a mixture solution. The mixture solution is then used to form different chitinous-alginate composite materials. In some embodiments, the mixture solution is cast into a coagulant to form chitinous-alginate composite fibers with optional additive such as vitamin E. The chitinous-alginate composite fibers with the optional additive can have improved strength, flexibility, elongation, moisture absorbency, surface area, and wound healing properties, etc. In a further aspect, methods of using the chitinous-alginate composite material with optional additive as wound dressing are disclosed.

Chitinous and Alginate

Alginate can be combined with the chitinous source material in a variety of suitable methods to form the chitinous-alginate solution. Alginic acid or alginate are available in filamentous, granular, or powder forms. In some embodiments, alginate is combined with the solid of the chitinous source material to form a combined solid mixture. Suitable ionic liquids or ion-containing solvent is then used to dissolve the combined solid mixture to form the chitinous-alginate solution. In further embodiments, alginate is introduced into the already formed chitinous solution to dissolve the alginate to form the chitinous-alginate solution. In further embodiments, the chitinous source material is introduced to an alginate solution that contains suitable ionic liquids or ion-containing solvent to form the chitinous-alginate solution. In further embodiments, a solution of alginate is combined with a solution of the chitinous source material to form the chitinous-alginate solution. The chitinous-alginate solution made from dissolving alginate in an chitinous IL solution are used in the following disclosure as an example; it is understood, however, that any of the methods listed above can similarly be used to form suitable chitinous-alginate solution. Optional additives can then be added into the chitinous-alginate solution to form a mixture solution that can be further processed to form composite materials such as fibers, films, membranes, granules, and filaments. For example, the chitinous-alginate solution with the optional additives is cast into a coagulant to form chitinous-alginate composite fibers. The composite fibers disclosed herein can comprise at least 80% by weight of combined chitinous and alginate components. The chitinous-alginate composite fibers can comprise an optional additive(s).

Chitin derived from crustaceans is available from suppliers as “pure chitin” and as “practical grade chitin.” These forms of chitin undergo a process similar to the Kraft Process for obtaining cellulose from wood or other sources of cellulose. During the process of preparing pure chitin and practical grade chitin, there is a breakdown of the polysaccharide chains such that the resulting chitin has a shorter chain length and therefore a lower average molecular weight than it had before it was processed. The composite fiber disclosed in the examples below uses HM chitin directly extracted from a chitinous biomass without substantially shortening the polysaccharide chains. As such, the fibers disclosed in the examples below comprise chitin that substantially retained the original full polysaccharide chain length and molecular weight. Although the composite fibers disclosed in the examples below uses HM chitin, it is understood that the commercially available “pure chitin” or “practical grade chitin” can also be used as chitinous source material. The composite chitinous-alginate fiber has a continuous and homogenous morphology even with the addition of the optional additive. Moreover the disclosed chitinous-alginate fiber can be substantially free of agents that are typically found in pure and practical grade chitin, such as methanesulfonic acid, trichloroacetic acid, dichloroacetic acid, formic acid, and dimethylacetamide. Although composite fiber is disclosed in the examples, it is understood that the chitinous-alginate solutions disclosed herein can be processed into other form of composite materials including fibers, films, membranes, granules, and filaments.

Additive

The unique property of the chitinous-alginate composite material disclosed herein provides an unique scaffold for adding additives that are otherwise difficult to be incorporated. For example, due to insolubility of Vitamin E in the aqueous media, consistent controlled release of vitamin has been difficult to achieve. Taepaiboon et al has attempted to use cellulose acetate nanofibers as a new Vitamin E releasing media according to published literature in “Vitamin-loaded electrospun cellulose acetate nanofiber mats as transdermal and dermal therapeutic agents of vitamin A acid and vitamin E,” Eur. J. Pharm. Biopharm. 2007, 67, 387-397. Cellulose acetate however, has limited use in medical related applications. In the examples disclosed below, vitamin E is added directly into the chitinous-alginate solution before casting the chitinous-alginate composite fiber. The chitinous-alginate composite fiber thus formed contains vitamin E without substantial compromise of the physical properties of the fibers and therefore provides the medical benefit of chitin, alginate as well as vitamin E simultaneously. Other suitable additives include vitamins, ionic liquid active pharmaceutical ingredients (IL-API), nutraceuticals, non-steroidal anti-inflammatory drugs, anesthetics, and analgesics.

In various examples disclosed herein, the contemplated chitinous-alginate fiber can contain an additive in an amount from about 0.01 to about 20 wt. %, from about 0.01 to about 18 wt. %, from about 0.01 to about 16 wt. %, from about 0.01 to about 14 wt. %, from about 0.01 to about 12 wt. %, from about 0.01 to about 10 wt. %, from about 0.01 to about 8 wt. %, from about 0.01 to about 6 wt. %, from about 0.01 to about 4 wt. %, from about 0.01 to about 2 wt. %, from about 0.01 to about 1 wt. %, or from about 0.01 to about 0.5 wt. %, of the composite fiber.

Ionic Liquids

ILs are useful in processes due to their non-volatility, solubilizing properties, recycling ability, and ease of processing (Rogers and Seddon, Science 2003, 302:792). ILs can often be viable alternatives to traditional industrial solvents comprising volatile organic compounds (VOCs). In particular, the use of ILs can substantially limit the amount of organic contaminants released into the environment. As such, ILs are at the forefront of a growing field known as “green chemistry.”

The ILs suitable for use in forming the chitinous-alginate solution comprise ionized species (i.e., cations and anions) and have melting points below about 150° C. For example, the ILs can be liquid at or below a temperature of about 150° C., about 100° C., or about 85° C., and at or above a temperature of about minus 100° C. or about minus 44° C. In some examples, a suitable IL can be liquid (molten) at a temperature of about minus 10° C. to about 150° C., about minus 4° C. to about 100° C., or about 25° C. to about 85° C. The term “liquid” describes a generally amorphous, non-crystalline, or semi-crystalline state. For example, while some structured association and packing of cations and anions can occur at the atomic level, an IL can have minor amounts of such ordered structures and are therefore not crystalline solids. The ILs can be fluid and free-flowing liquids or amorphous solids such as glasses or waxes at temperatures at or below about 150° C. In some embodiments, the ILs can be fluid and free-flowing liquids or amorphous solids such as glasses or waxes at temperatures at or below about 100° C. In particular examples described herein, the ILs are liquid at the temperature at which they are applied or used.

ILs suitable for use herein can be hydrophilic or hydrophobic and can be substantially free of water, substantially free of alcohol-miscible organic solvent, and/or nitrogen-comprising base. By “substantially free” is meant less than about 10 wt. %, e.g., less than about 10, 9, 8, 7, 6, 5, 4, 3, 2, or 1 wt. % of the identified component. Contemplated organic solvents of which the ionic liquid can be substantially free of include solvents such as diethyl sulfoxide, dimethyl formamide, acetamide, hexamethyl phosphoramide, water-soluble alcohols, ketones or aldehydes such as ethanol, methanol, 1- or 2-propanol, tert-butanol, acetone, methyl ethyl ketone, acetaldehyde, propionaldehyde, ethylene glycol, propylene glycol, the C₁-C₄ alkyl and alkoxy ethylene glycols and propylene glycols such as 2-methoxyethanol, 2-ethoxyethanol, 2-butoxyethanol, diethyleneglycol, and the like.

It should be appreciated that some water may be present in the ILs (and ion containing solvents) during use. Such residual amounts should be taken into account even though a system is described to be “substantially free of” water. The same meaning is intended regarding the presence of a nitrogen-comprising base, alcohol, or other miscible organic solvent.

In other examples, the IL can contain processing aids (as discussed elsewhere herein) to assist in chitin and alginate dissolution. These processing aids can be used in amounts up to 50% by weight of the IL. The processing aids can be used to modify the viscosity and/or melting point of the IL, and/or to improve the solubility of the chitin and alginate. Processing aids can also be used to improve the precipitation and removal of various undesired components like cellular debris, lipids, proteins, mineral salts, and the like. Examples of processing aids suitable for use herein include the organic solvents mentioned above and water.

Some specific examples of ILs that can be used to form chitinous-alginate solution herein are disclosed in Qin et al., WO2010/141470, entitled “Process for forming films, fibers, and beads, from Chitinous Biomass”; Xie et al., “Chitin and chitosan dissolved in ionic liquids as reversible sorbents of CO₂,” Green Chem 8:630-633 (2006); Prasad et al., “Weak gel of chitin with ionic liquid, 1-allyl-3-methylimidazolium bromide,” Int J Biol Macromol 45:221-225 (2009); and Qin et al., “Dissolution or extraction of crustacean shells using ionic liquids to obtain high molecular weight purified chitin and direct production of chitin films and fibers,” Green Chem 12:968-971 (2010), which are each incorporated by reference herein for their teachings of suitable ILs. Other examples of suitable ILs that can be used herein are disclosed in U.S. Pat. No. 6,824,599 and Swatloski et al., J Am Chem Soc 2002, 124:4974-4975, U.S. application Ser. Nos. 13/375,245; 13/505,323; and 13/428,786 and U.S. Provisional Application Nos. 61/674,979 and 61/764,770, which are incorporated by reference herein for their teachings of suitable ILs. Other specific examples of suitable ILs are disclosed herein.

In some embodiments, the IL can actually be a mixture of ILs, prepared by reacting IL precursors in one-pot to form the ILs. An IL precursor is a compound that can form any of the cations or anions disclosed herein. In this sense the ILs can be crude mixtures, containing different types of cations and/or different types of anions, and some organic or water solvent. The use of crude IL mixtures to dissolve polymers is taught in WO2011/056924, which is incorporated by reference herein in its entirety for its teachings of polymer dissolution using IL mixtures.

Ion-Containing Solvents

Also disclosed herein is the use of ion-containing solvents to dissolve chitinous source material and alginate. The ion-containing solvents can comprise ion pairs, eutectics, liquid co-crystals, or non-stoichiometric ionic liquids. Such solvents comprise cations and anions as well as complexed non-ionized species such as coordinated or hydrogen-bonded complexes where an acid molecule is associated with the base through hydrogen bonding.

Cations

As noted, ILs and ion-containing solvents contain one or more types of cations and one or more types of anions. While specific ILs are discussed above and elsewhere herein, other ILs can be used by combining the various cations and anions that follow, with optional inclusion of non-ionized acid or base. But depending on the particular ion and ratios thereof, the resulting product can be an IL or an ion-containing solvent (i.e., eutectic, ion pair, or liquid co-crystal), any of which can be suitable for use in the disclosed methods.

In many examples, the cation can comprise a linear, branched, or cyclic heteroalkyl unit. The term “heteroalkyl” refers to a cation as disclosed herein comprising one or more heteroatoms chosen from nitrogen, oxygen, sulfur, boron, or phosphorous capable of forming a cation. The heteroatom can be a part of a ring formed with one or more other heteroatoms, for example, pyridinyl, imidazolinyl rings, that can have substituted or unsubstituted linear or branched alkyl units attached thereto. In addition, the cation can be a single heteroatom wherein a sufficient number of substituted or unsubstituted linear or branched alkyl units are attached to the heteroatom such that a cation is formed. For example, the cation Cₙ alkyl-methylimidazolium [Cₙmim] where n is an integer of from 1 to 8 can be used. Preferably, the cation C₁₋₄ alkyl-methylimidazolium [C₁₋₄mim] can be used. [Amim] is an allyl methylimidazolium ion and is suitable for use herein. [C₂C₂Im] is diethylimizazolium ion and is suitable for use herein.

Other non-limiting examples of heterocyclic and heteroaryl units that can be alkylated to form cationic units include imidazole, pyrazoles, thiazoles, isothiazoles, azathiozoles, oxothiazoles, oxazines, oxazolines, oxazaboroles, dithiozoles, triazoles, selenozoles, oxahospholes, pyrroles, boroles, furans, thiophenes, phospholes, pentazoles, indoles, indolines, oxazoles, isothirazoles, tetrazoles, benzofurans, dibenzofurans, benzothiophenes, dibenzothoiphenes, thiadiazoles, pyrdines, pyrimidines, pyrazines, pyridazines, piperazines, piperidines, morpholines, pyrans, annolines, phthalazines, quinazolines, and quinoxalines.

The following are examples of heterocyclic units that are suitable for forming a cyclic heteroalkyl cation unit of suitable ILs or ion-containing solvents.

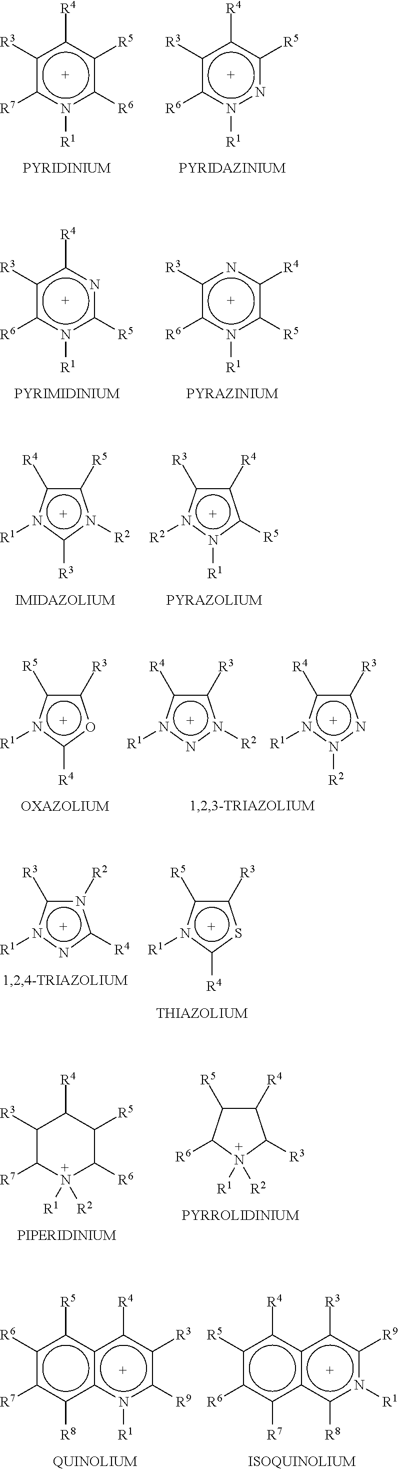

wherein R¹ and R² are independently a C₁-C₆ alkyl group or a C₁-C₆ alkoxyalkyl group, and R³, R⁴, R⁵, R⁶, R⁷, R⁸, and R⁹ (R³-R⁹), when present, are independently H, a C₁-C₆ alkyl, a C₁-C₆ alkoxyalkyl group, or a C₁-C₆ alkoxy group. In other examples, both R¹ and R² groups are C₁-C₄ alkyl, with one being methyl, and R³-R⁹, when present, are H. Exemplary C₁-C₆ alkyl groups and C₁-C₄ alkyl groups include methyl, ethyl, propyl, iso-propyl, butyl, sec-butyl, iso-butyl, pentyl, iso-pentyl, hexyl, 2-ethylbutyl, 2-methylpentyl, and the like. Corresponding C₁-C₆ alkoxy groups comprise the above C₁-C₆ alkyl group bonded to an oxygen atom that is also bonded to the cation ring. An alkoxyalkyl group comprises an ether group bonded to an alkyl group, and here comprises a total of up to six carbon atoms. It is to be noted that there are two isomeric 1,2,3-triazoles. In some examples, all R groups not required for cation formation can be H.

The phrase “when present” is often used herein in regard to substituent R group because not all cations have all of the numbered R groups. All of the contemplated cations comprise at least four R groups, which can, in various examples, be H.

In one example, all R groups that are not required for cation formation; i.e., those other than R¹ and R² for compounds other than the imidazolium, pyrazolium, and triazolium cations shown above, are H. Thus, the cations shown above can have a structure that corresponds to a structure shown below, wherein R¹ and R² are as described before.

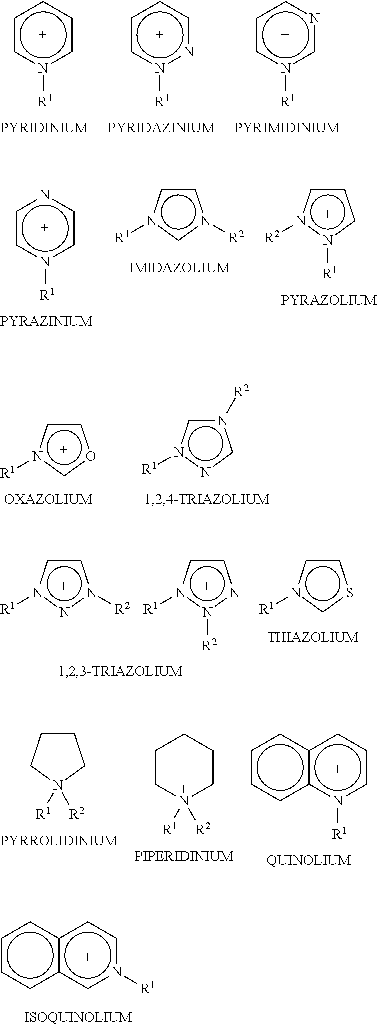

A cation that comprises a single five-membered ring that is free of fusion to other ring structures is also a suitable cation for the compositions and methods disclosed herein.

In additional examples, a suitable cation can correspond in structure to a formula shown below:

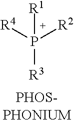

wherein R¹, R², R³, and R⁴, when present, are independently a C₁-C₁₈ alkyl group or a C₁-C₁₈ alkoxyalkyl group.

Still further examples of suitable cations include ammonium, alkoxyalkyl imidazolium, alkanolyl substituted ammonium, alkoxyalkyl substituted ammonium, aminoalkyl substituted ammonium.

Anions

The choice of the anion can be particularly relevant to the rate and level of chitinous source material and alginate dissolution. While not wishing to be bound by theory, the primary mechanism of solvation of polysaccharides by an IL or ion-containing solvent is the anion's ability to break the extensive hydrogen-bonding networks by specific interactions with hydroxyl groups. For example, it is believed that the dissolution of chitin is enhanced by increasing the hydrogen bond acceptance and basicity of the anion. Anions that lower the hydrogen bond bascicity (i.e., that add hydrogen bond donors) in too great of an excess should be avoided. Anions that also form less viscous ILs or ion-containing liquids are also preferred.

Accordingly, preferred anions are substituted or unsubstituted acyl units R¹⁰CO₂, for example, formate HCO₂, acetate CH₃CO₂, proprionate, CH₃CH₂CO₂, butyrate CH₃CH₂CH₂CO₂, and benzylate, C₆H₅CO₂; substituted or unsubstituted sulfates: (R¹⁰O)S(═O)₂O; substituted or unsubstituted sulfonates R¹⁰SO₃, for example (CF₃)SO₃; substituted or unsubstituted phosphates: (R¹⁰O)₂P(O)O; and substituted or unsubstituted carboxylates: (R¹⁰O)C(═O)O. Non-limiting examples of R¹⁰ include hydrogen; substituted or unsubstituted linear, branched, and cyclic alkyl; substituted or unsubstituted linear, branched, and cyclic alkoxy; substituted or unsubstituted aryl; substituted or unsubstituted aryloxy; substituted or unsubstituted heterocyclic; substituted or unsubstituted heteroaryl; acyl; silyl; boryl; phosphino; amino; thio; and seleno. In preferred examples, the anion is C₁₋₆ carboxylate. Carboxylate anions that contain 1-6 carbon atoms (C₁-C₆ carboxylate) and are illustrated by formate, acetate, propionate, butyrate, hexanoate, maleate, fumarate, oxalate, lactate, pyruvate, and the like.

Other suitable anions are halogen (fluoride, chloride, bromide, or iodide), perchlorate, a pseudohalogen such as thiocyanate and cyanate, or C₁-C₆ carboxylate. Pseudohalides are monovalent and have properties similar to those of halides (Schriver et al., Inorganic Chemistry, W. H. Freeman & Co., New York, 1990, 406−407). Pseudohalides include the cyanide (CN⁻), thiocyanate (SCN⁻), cyanate (OCN⁻), fulminate (CNO⁻), and azide (N₃⁻) anions. Still other examples of suitable anions are persulfate, sulfate, sulfites, phosphates (e.g., (CH₃)₂PO₄), phosphites, nitrate, nitrites, hypochlorite, chlorite, perchlorate, bicarbonates, and the like, including mixtures thereof.

Still further examples of suitable anions are deprotonated amino acids, for example, Isoleucine, Alanine, Leucine, Asparagine, Lysine, Aspartic Acid, Methionine, Cysteine, Phenylalanine, Glutamic Acid, Threonine, Glutamine, Tryptophan, Glycine, Valine, Proline, Selenocysteine, Serine, Tyrosine, Arginine, Histidine, Ornithine, Taurine.

It is also contemplated that other anions, though not preferred, can still be used in some instances. However, in these instances, higher concentrations, longer mixing times, and higher temperatures can be required. One can use CO₃²; NO₂⁻, NO₃⁻, SO₄², CN⁻, arsenate(V), AsX₆; AsF₆, and the like; stibate(V) (antimony), SbX₆; SbF₆, and the like.

Processing Aids

Processing aids can be added to the IL or ion-containing solvent in order to help lower the cost, lower the viscosity, aid in recycling, stiochiometrically or nonstoichiometrically interact with chitinous source material, alginate, and other components to increase dissolution, facilitate disintegration, cleave bonds, and for derivatization and other treatments. Any processing aid can be used in these methods as long as the ionic liquid media does not inactivate the processing aid. Carboxylate salts such as sodium, potassium, ammonium, and choline acetates can be added to the ionic liquid mixtures to facilitate dissolution. Some other examples of processing aids, include but are not limited to, catalysts, metal salts, polyoxymetalates (POMs) (e.g., H₅[PV₂Mo₁₀O₄₀]), anthraquinone, enzymes, and the like. It is also possible to add solvents to the ionic liquid mixtures to aid in dissolution and processing. For example, ethanol, glycol, polyethylene glycol, DMSO, DMF, polyvinylalcohol, polyvinylpyrrolidone, furan, pyridine and other N containing bases, and the like can be added. In some examples the ionic liquid mixtures can be mixed with polyalkylene glycols as disclosed in WO09/105236, which is incorporated by reference herein for its teaching of fractioning polymers and their use in ionic liquids. In further examples, the following ammonium salts can be added to the ionic liquids to improve dissolution: Bu₄NOH, Bu₄N(H₂PO₄), Me₄NOH, Me₄NCl, Et₄NPF₆, and Et₄NCl. Any of these processing aids can be added in amounts of up to about 50 wt. % of the IL or ion-containing solvent, e.g., from about 1 to about 10 wt. %, from about 10 to about 40 wt. %, from about 20 to about 30 wt. %, from about 20 to about 50 wt. %, or from about 40 to about 50 wt. %.

### SPECIFIC EXAMPLES

Suitable ILs or ion-containing solvents for the disclosed chitinous-alginate solution can comprise any of the cations and anions disclosed herein. For example, the composition can comprise a 1-alkyl-3-methylimidazolium halide or a 1-alkyl-3-methylimidazolium C₁₋₆ carboxylate (e.g., a 1-alkyl-3-methylimidazolium C₁₋₆ acetate). Some further specific examples include, but are not limited to, 1-ethyl-3-methylimidazoium chloride, 1-butyl-3-methylimidazolium chloride, 1-ethyl-3-methylimidazolium acetate, 1,3-diethylimidazolium acetate, 1,3-dimethylimidazolium acetate, allylmethylimidazolium chloride, allylbutylimidazolium chloride, diallylimidazolium chloride, allyloxymethylimidazolium chloride, allylhydroxyethylimidazolium chloride, allylmethylimidazolium formate, allylmethylimidazolium acetate, benzylmethylimidazolium chloride, bis(methylimidazolium)sulfoxide chloride, ethylmethylimidazolium benzoate, ethylmethylimidazolium triflate, ethylmethylimidazolium chloride, ethylmethylimidazolium acetate, ethylmethylimidazolium xylenesulfonate, ethylmethylimidazolium methylphosphonate, propylmethylimidazolium formate, butylmethylimidazolium chloride, butylmethylimidazolium chloride+FeCl₃, butylmethylimidazolium MeSO₄, butylmethylimidazolium (CN₂)N—, butyl-2,3-dimethylimidazolium chloride, methylhydroxyethylimidazolium chloride, N,N′-dimethylimidazolium chloride, N,N′-dimethylimidazolium mesylate, N,N′-dimethylimidazolium acetate, 1-(2-hydroxylethyl)-3-methylimidazoium chloride, 1-methyl-3-(4-vinylbenzyl)imidazolium chloride, 3,3-ethane-1,2-dylbis(methylimidazolium)dichloride, 3,3-ethane-1,2-dylbis(methylimidazolium)dichloroaluminate, 1-vinyl-3-(4-vinylbenzyl)imidazolium chloride, diethyl N-methyl-N-(2-methoxyethyl)ammonium Tf₂N, hydroxybutyl trimethylammonium carbamate, nitronium Tf₂N, tetrabutylammonium benzoate, tetrabutylammonium, dodecylbenzenesulfonate, tetrabutylammonium hydroxide, tetrabutylammonium xylenesulfonate, phenyltributylammonium xylenesulfonate, allylmethylpyridinium chloride, benzylpyridinium chloride, butylmethyl pyrrolidinium 4-hydroxybenzenesulfonate, ethylpyridinium bromide, trihexyltetradecylphosphonium xylenesulfonate, choline chloride+urea, choline chloride+ZnCl₂, and 1-methyl-3 butyl-imidazolium thioacetate.

Some additional examples of ILs include, but are not limited to, the following quaternary ammonium salts: Bu₄NOH, Bu₄N(H₂PO₄), Me₄NOH, Me₄NCl, Et₄NPF₆, and Et₄NCl.

### Chitinous-Alginate Fibers

In various examples disclosed herein, the contemplated chitinous-alginate solution in the IL or ion-containing solvent can contain chitinous source material and alginate in a total amount of chitinous source material and alginate from about 0.55 to about 10 wt. %, from about 0.55 to about 9 wt. %, from about 0.55 to about 8 wt. %, from about 0.55 to about 7 wt. %, from about 0.55 to about 6.0 wt. %, from about 0.55 to about 5 wt. %, from about 0.55 to about 4.0 wt. %, from about 0.55 to about 3 wt. %, or from about 0.55 to about 1.0 wt. % of the mixture. The solution can contain chitinous source material in an amount of from about 0.5 to about 9.95 wt. %, from about 0.5 to about 9.0 wt. %, from about 0.5 to about 8.0 wt. %, from about 0.5 to about 7.0 wt. %, from about 0.5 to about 6.0 wt. %, from about 0.5 to about 5.0 wt. %, from about 0.5 to about 4.0 wt. %, from about 0.5 to about 3 wt. %, from about 0.5 to about 2.0 wt. %, or from about 0.5 to about 1.0 wt. % of the mixture.

The solution can contain alginate in an amount of from about 0.5 to about 9.5 wt. %, from about 0.4 to about 8.0 wt. %, from about 0.35 to about 7.0 wt. %, from about 0.3 to about 6.0 wt. %, from about 0.25 to about 5 wt. %, from about 0.2 to about 4 wt. %, from about 0.15 to about 3 wt. %, from about 0.1 to about 2.0 wt. %, or from about 0.05 to about 0.5 wt. % of the mixture. In the chitinous-alginate solution, the weight ratio between the chitinous component and the alginate component is at least 1:1, at least 1.5:1, at least 2:1, at least 2.5:1, at least 3:1, at least 3.5:1, at least 4:1, at least 5:1, at least 6:1, at least 7:1, at least 8:1, at least 9:1 or at least 10:1.

Higher concentrations tend to produce solutions that are viscous and difficult to process such as spinning fibers from. By using processing aids as discussed herein, however, higher concentrations of chitinous-alginate solution can be obtained, e.g., from greater than about 5, 10, 15, or even 20 wt. %. A processing aid can already be present in the IL or ion-containing solvent or can be added after the chitinous source material and alginate dissolved. The disclosed chitinous-alginate solution can also comprise one IL or ion-containing solvent or mixtures of two or more ILs or ion-containing solvents in any suitable combination with or without processing aids.

Various processing methods can be used to dissolve the chitinous source material and alginate. For example, the mixed solid of chitinous source material and alginate can be contacted with one or more ILs or ion-containing solvents by submerging the solid into the liquid. Suitable ILs or ion-containing solvents for dissolving the chitinous source material and alginate are discussed herein and include 1-ethyl-3-methylimidazolium acetate ([C₂mim]OAc, also known as [Emim][OAc]), 1-ethyl-3-methylimidazoium chloride ([C₂mim]Cl), 1-butyl-3-methylimidazolium chloride ([C₄mim]Cl, 1,3-diethylimidazolium acetate [C₂C₂Im]OAc, 1,3-dimethylimidazolium acetate [C₁C₁Im]OAc). The other ionic liquids (cation and anion combinations contemplated herein) can also be used.

Optionally, the dissolution of chitinous source material and alginate can be aided by mechanically agitating the mixture. For example, the mixture can be stirred, blended, or sonicated to form a slurry. In some aspects, the mixture is agitated at a low temperature or at room temperature. In other aspects, the mixture is agitated at an elevated temperature. In further aspects, the composition can be cooled or heated at a temperature effective for dissolving the chitin and alginate in the ionic liquid(s), for example, from about 0° C. to about 250° C., from about 0° C. to about 120° C., from about 40° C. to about 120° C., from about 80° C. to about 120° C.

In some aspects, the mixture can be irradiated with microwaves, infrared, or ultrasound irradiation, and/or other external sources of energy supply such as heat. It is known from the recent literature concerning organic synthesis that the reaction times of organic reactions are remarkably reduced when the energy necessary for the occurrence of the reaction is introduced to the system by using microwave irradiation. There is a wide and continuously increasing literature available in the area of using microwave techniques in organic synthesis. An example of a short summary article of this topic was published by Mingos in 1994 (“Microwaves in chemical synthesis,” Chem. Indus. 596-599 (1994)). Loupy et al. have recently published a review concerning heterogenous catalysis under microwave irradiation (Loupy, “New solvent-free organic synthesis using focused microwave,” Synthesis 1213-1234 (1998)). Another representative article of the area is published by Strauss as an invited review article (“A combinatorial approach to the development of environmentally benign organic chemical preparations,” Aust. J. Chem. 52:83-96 (1999)).

Because of their ionic nature, ILs and ion-containing solvents are excellent media for utilizing microwave techniques. The commonly used frequency for microwave energy is 2.45 GHz. In the disclosed methods, the frequency for microwave energy can be reduced. In some aspects, the lower frequency results in higher dissolution of the chitinous source material and alginate. For example, the frequency for microwave energy can be less than 2.0 GHz, less than 1.5 GHz, or less than 1.0 GHz. In some aspects, the frequency for microwave energy is 990 MHz or less, 980 MHz or less, 970 MHz or less, 960 MHz or less, 950 MHz or less, 940 MHz or less, 930 MHz or less, 920 MHz or less, 915 MHz or less, 910 MHz or less, or 900 MHz or less. In some aspects, the frequency for microwave energy is 915 MHz.

Any processing time can be used to get the chitin and alginate to at least partially dissolve in the mixture, for example from seconds to hours, such as from 1 to 16 hours, 1 to 12 hours, or from 1 to 5 hours. At lower temperatures, the processing time is longer. At higher temperatures, with mechanical agination, or under microwave irradiation, the processing time is shorter.

The chitinous-alginate solution can then be processed into chitinous-alginate composite materials including fibers, filaments, films, membranes, and granules. For example, the chitinous-alginate solution can be drawn into fibers by casting/spinning the chitinous-alginate solution into a non-solvent (also called a coagulant). The non-solvent can be water, a C₁-C₁₂ linear or branched alcohol, ketone (e.g., acetone or methylethylketone), or other organic solvent not suitable for dissolving chitin and alginate. In one example, the non-solvent is water. In another example, the non-solvent is a C₁-C₄ linear or branched alcohol, for example, methanol, ethanol, propanol, iso-propanol, butanol, sec-butanol, iso-butanol, or tert-butanol. In one example, ethanol is used as the non-solvent. In a further example, a mixture of water and a C₁-C₄ linear or branched alcohol can be used as a non-solvent, for example, water/methanol, water/ethanol, and the like. For this example, any ratio of water to solvent can be used, for example, from about 5:95 water/solvent to 95:5 water/solvent.

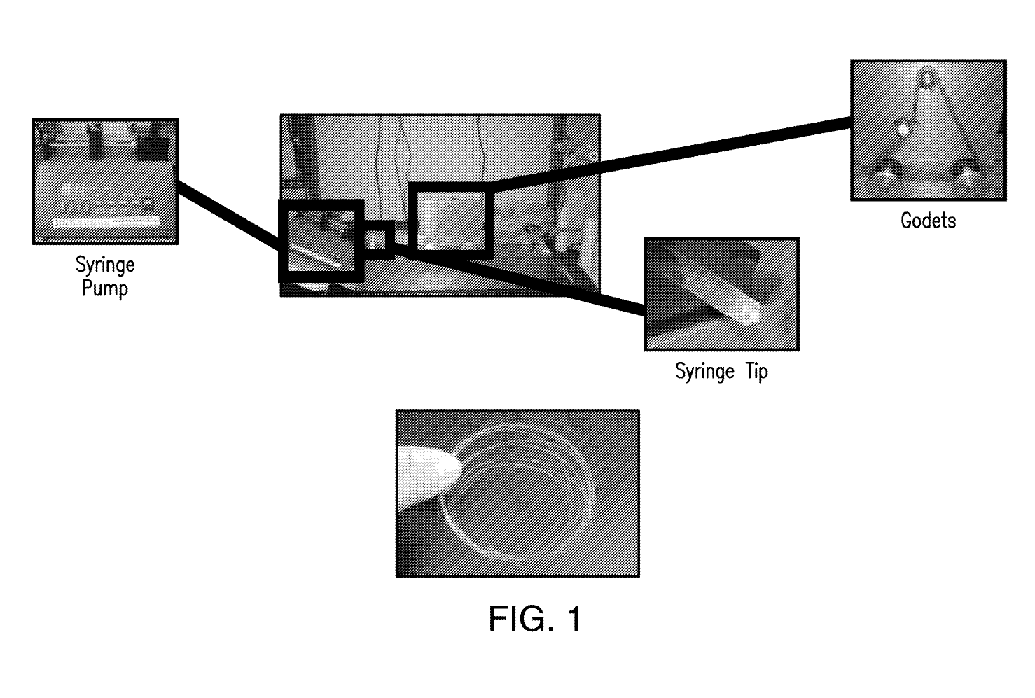

A dry jet wet spinning method as described for producing cellulose fibers from IL solution (Sun et al., J. Mater. Chem. 18:283-290, 2008, which is incorporated herein for its teachings of fiber spinning techniques) is adapted to produce the chitinous-alginate composite fiber disclosed herein. Specifically, a schematic representation of a dry jet wet spinning set up 100 is shown in FIG. 1. In the set up, syringe pump 102 casts chitinous-alginate solution into a coagulant bath 104. Upon contacting the coagulant, the chitinous-alginate solution forms a fiber, one end of which is drawn with a godets 106 continuously to collect the formed fiber. Upper right side is an enlarged view of the back side of the godets, showing the mechanical details. Lower left side corner is an enlarged view of the syringe that is connect to and operated by the syringe pump. The syringe contains straw colored chitinous-alginate solution 108. The tip of the syringe is positioned above the water bath 104. The bottom of the figures shows a bundle of chitinous-alginate fibers 110 formed with the set up. The speed of the spinning godets in combination with the pumping speed of the syringe pump influence the diameter or thickness of the chitinous-alginate fiber formed. By adjusting viscosity of the chitinous-alginate solution and the spinning and pumping speeds, chitinous-alginate fiber with different diameters are obtained. The chitinous-alginate composite fiber can be used to form fiber containing sheets or mats. Both surface and morphology studies demonstrated that produced chitin, chitin-alginate and Vitamin E-loaded fibers exhibited a homogeneous morphology, indicating blend homogeneity between all three components. All produced fibers were continuous, with aligned fiber orientation.

In various examples disclosed herein, the contemplated chitinous-alginate composite fiber contain chitinous source material and alginate in a total amount of at least 80 wt. %, at least 82 wt. %, at least 84 wt. %, at least 86 wt. %, at least 88 wt. %, at least 90 wt. %, at least 92 wt. %, at least 94 wt. %, at least 96 wt. %, at least 98 wt. %, at least 99 wt. %, or at least 99.5 wt. % of the composite. The weight ratio between the chitinous source material component and the alginate component in the composite fiber is at least 1:1, at least 1.5:1, at least 2:1, at least 2.5:1, at least 3:1, at least 3.5:1, at least 4:1, at least 5:1, at least 6:1, at least 7:1, at least 8:1, at least 9:1 or at least 10:1.

In some embodiments, the chitinous component in the chitinous-alginate composite fiber described herein has a higher molecular weight than commercially available chitinous products. The chitinous-alginate composite fiber thus formed has stronger profiles. In some aspects, the composite chitinous-alginate fibers have tenacity of 195 MPa or more, 200 MPa or more, 205 MPa or more, 210 MPa or more, or 215 MPa or more. The stress measurements disclosed herein were taken on a length of 10 cm using a MTSQ25 machine attached with a specially designed pneumatic grip suitable for thin and flexible fiber testing. A load cell of 22.4 newton capacity (5 lb) was used for load measurement. The cross head speed was maintained at 1.27 mm min⁻¹ and the test data in terms of stress and strain were obtained using a data acquisition system. The tenacity of the composite fiber with or without the optional additive is at least 5% more, at least 6% more, at least 7% more, at least 8% more, at least 9% more, at least 10% more, at least 12% more, or at least 15% more than the tenacity of alginate fibers.

Further, the composite fibers described herein has higher liner mass density than alginate fibers or chitosan fibers reported in the literature. In some aspects, the composite chitinous-alginate fibers have liner mass density of 200 g/m or more, 210 g/m or more, 220 g/m or more, 240 g/m or more, 250 g/m or more, 260 g/m or more, 270 g/m or more, 280 g/m or more, 300 g/m or more, 310 g/m or more, 320 g/m or more, or 330 g/m or more. The linear density of the composite alginate-chitin fiber is at least 5 times, at least 6 times, at least 7 times, at least 8 times, at least 9 times, at least 10 times, at least 11 times, at least 12 times, at least 13 times, at least 14 times, or at least 15 times more than the liner mass density of alginate or chitosan fibers.

Additionally, the composite fibers described herein has lower percentage of elongation when stretched when compared to literature reported value of alginate fibers or chitosan fibers. In some aspects, the composite chitinous-alginate fibers have a elongation of about 10% or less, about 9% or less, about 8% or less, about 7% or less, or about 6% or less. The percent of elongation of the composite chitinous-alginate fiber is at least 30% less, at least about 40% less, at least about 50% less, at least about 75% less, at least about 100% less, at least about 125% less, at least about 150% less, at least about 175% less, at least about 200% less, at least about 250% less, at least about 300% less, at least about 350% less, or at least about 500% less than the elongation of alginate or chitosan fibers.

Additionally, the composite fibers described herein has lower percentage of residual ionic liquid. For example, the disclosed composite fibers can have less than about 5 wt. %, less than about 2 wt. %, less than about 1 wt. %, less than about 0.5 wt. %, or less than about 0.1 wt. % ionic liquid based on the weight of the fiber.

The chitinous-alginate composite fibers are produced with consistent reproducibility. The composite fibers are biodegradable, antimicrobial, stable and easy to store.

### Wound Patch Chitinous-Alginate Fibers

The fibers described herein have been used to prepare the wound patches for wound healing applications. The chitinous-alginate patch performed better than other products used for wound care, for example, the OPSITE™ dressing marketed by Smith and Nephew, or products described in the literature like chitosan fibers, alginate fibers, chitosan-alginate membranes (see Wang et al. “Chitosan-Alginate PEC Membrane as a Wound Dressing: Assessment of Incisional Wound Healing”. J. Biomed. Mater. Res 2002, 63, 601-618.). The chitinous-alginate composite fibers with or without the optional vitamin E additive are shown to be effective and versatile wound dressings that displays accelerated wound healing properties with less or no reapplication required.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1

### Fiber Preparation

The extraction of high molecular weight chitin was performed using a modified published procedure disclosed by Qin et al. in “Dissolution or Extraction of Crustacean Shells Using Ionic Liquids to Obtain High Molecular Weight Purified Chitin and Direct Production of Chitin Films and Fibers”. Green Chem. 2010, 12, 968-971, by direct dissolution of raw biomass in the IL followed by subsequent precipitation. Specifically, shrimp and crab were thawed and carefully peeled to make sure no obvious meat was left. The shells were washed three times with tap water and then dried in an oven (Precision Econotherm Laboratory, Winchester, Va.) at 80° C. for 2 days. The dried shells were ground for 1 min using a Janke & Kunkel mill (Ika Labortechnik, Wilmington, N.C.) and separated using brass sieves (Ika Labortechnik, Wilmington, N.C.) with pore sizes ranging from 0.125 mm to 1 mm, into particle sizes of 0.125-0.5 mm.

Ionic liquid [C₂mim]OAc (purity ≧90%), also known as 1-ethyl-3-methylimidazolium acetate or [Emim][OAc] were obtained from Iolitec (Tuscaloosa, Ala.) and dried in a vacuum oven at about 70° C. for 20 h before use. Deionized (DI) water was obtained from a commercial deionizer Culligan, Northbrook, Ill.) with specific resistivity of 17.25 MΩ cm at 25° C. Dimethyl sulfoxide (DMSO) (≧99.6%) was purchased from Aldrich (St. Louis, Mo.) and used as received.

Approximately 1.2 g of dried, ground shrimp shells (as above) was mixed with 62 g of dried [Emim]OAc in an Erlenmeyer flask. The mixture was heated in a domestic microwave oven (Emerson MW8999SB, Emerson Radio Corp, Moonachie, N.J.)) using 2-3 s pulses at full power for 2-5 minutes. Care was taken to avoid overheating the IL. Between each pulse, the vial was removed, the mixture was manually stirred by a glass rod, and then replaced in the microwave. After total irradiation time of 2-5 min, the undissolved residues were removed via centrifugation.

The clear IL solution was poured slowly into a beaker containing 400 mL of coagulating solvent (DI water) and white flocs formed immediately. The mixture was then stirred at room temperature for 3-12 h and poured into four 50 mL plastic test tubes for centrifugation. After centrifugation at 100×g for 10 min, the settled flocs were washed with DI water (3-15×100 mL), and dried at 80° C. for 20 h in the oven to produce pure chitin. The pure chitin thus obtained has higher molecular weight (HW chitin) than commercially available chitin as discussed in copending U.S. provisional application 61/674,979, incorporated herein by reference. The dried HW chitin were ground for 1 min using a Janke & Kunkel mill (Ika Labortechnik, Wilmington, N.C.) and separated using brass sieves (Ika Labortechnik, Wilmington, N.C.) with pore sizes ranging from 0.125 mm to 1 mm, into particle sizes of 0.125-0.5 mm.

Approximately 0.178 g of dried, ground HW chitin (as above) was suspended in 10 g dried [Emim]OAc in a 20 mL vial. The suspension was heated in an oil bath (90-100° C.) until the dissolution was complete to form a chitin solution with 1.75 wt. % chitin in respect to the IL. Alternatively, the suspension can be heated in a domestic microwave oven using 3 s pulses at full power for 2-5 min till complete dissolution was reached. Between each pulse, the vial was removed; the mixture was manually stirred by a glass rod and then replaced in the microwave. Once the HW chitin dissolved, the alginic acid (0.06 g) was added and the mixture was magnetically stirred to ensure solution homogeneity. Complete dissolution of the biopolymers was monitored by removing a drop of the mixture and placing it in between two pieces of closely contacted glass slides for observation of any undissolved residue using an optical microscope (Reichert Stereo Star Zoom 580, Depew, N.Y.). The pure chitin and alginate powders (ca. 0.2 g) were found to be completely dissolved in 10 g of [Emim]OAc to form a clear chitinous-alginate mixture.

The chitinous-alginate solution was used to produce chitinous-alginate fibers using a dry-jet wet spinning method illustrated in FIG. 1 above. To convert the alginic acid to alginate, a saturated solution of calcium carbonate in DI water was used as the coagulant. After centrifugation, where gas bubbles were completely removed, each chitinous-alginate sample was carefully loaded into a 10-mL syringe which was then attached to a syringe pump (Model No. NE-1010, New Era Pump Systems, Inc, Farmingdale, N.Y.) to be pumped at a rate of 1.5 mL/min. Each solution was extruded into a 0.6 m long water bath after a 5 cm air gap. Each chitinous-alginate filament was led through the first two steps of the godets, and then wound onto the take-up spool. An extrusion rate of 1.5 mL/min was used. The voltage settings for godets and take-up spool were 2-2.3 V and 2.9 V, respectively. The produced fibers were soaked in a saturated solution of CaCO₃ for 5-10 min, and then placed into DI water that was subsequently changed several times to remove the IL. After soaking the fibers overnight in DI, the fibers were dried for 24 h in the air. A bundle of dried composite fiber is shown at the bottom of FIG. 1.

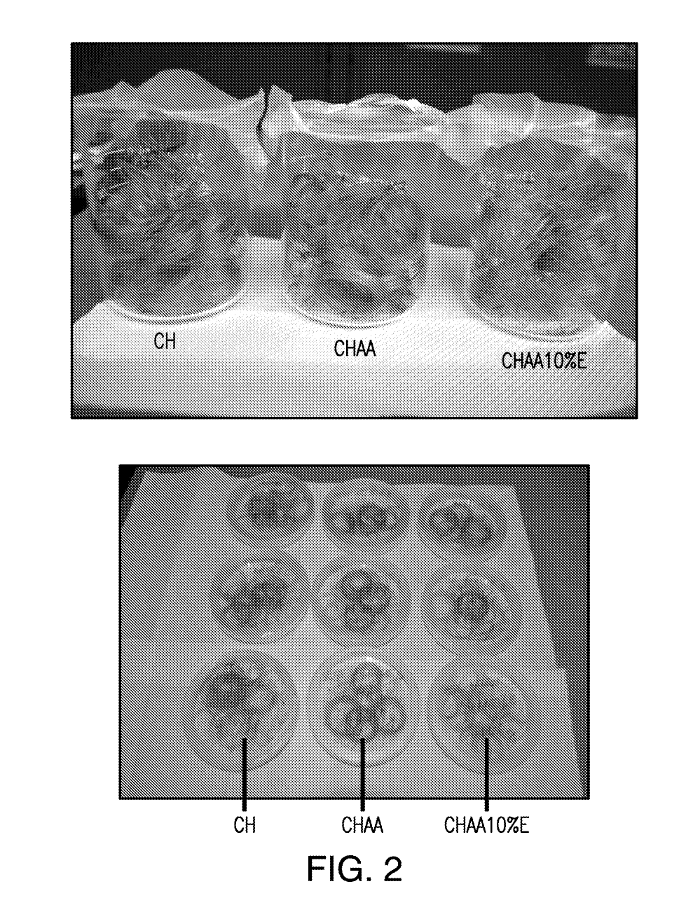

In an alternative embodiment, prior to the fiber formation, vitamin E (0.0089 g, 0.0178 g, or 0.0356 g) was added to the clear chitinous-alginate IL solution to form a homogenous chitinous-alginate vitamin E solution (5, 10, or 20 wt %). The solution was then centrifuged for ten minutes to degas, and allowed to stay in the oven at 80° C. for an hour. Vitamin E was chosen as an additive as it possesses an anti-oxidation property that helps to stabilize cell membranes, including cells of the inflammatory process. Vitamin E is also believed to have a protective effect against buildup of arterial plaque. The dry jet wet spinning method is then used to produce chitinous-aliginate vitamin E fibers (CHAAE5, CHAAE10, or CHAAE20), using the same condition as described above. Chitin fiber without alginate and vitamin was similarly produced for comparison purpose. FIG. 2 are photos of chitin fibers (CH), chitinous-alginate fibers (CHAA), and chitinous-alginate-vitamin E fibers (CHAA10% E) showing similar morphology.

### Example 2

### Fiber Characterization

Various aspects of the chitinous-alginate and chitinous-alginate-vitamin E fibers were characterized and the results discussed below

Determination of Vitamin E Amount in the Above Formed Fibers

To determine the amount of Vitamin E embedded, the CHAAE5, CHAAE10, or CHAAE20 fibers from example 1 above were treated with the medium in which vitamin E was soluble and could freely elute (methanol). Chitinous-alginate-vitamin E fibers CHAAE5, CHAAE10, or CHAAE20 were extensively stirred for 48 hours at room temperature to crash the fibers, and the suspension was filtered through a syringe filter to remove the undissolved particles. The filtrates were then analyzed by UV-Vis and the absorbance at 292 nm was selected for analysis and determination of amount of vitamin E present. The data is presented in Table 1. The amount of vitamin E determined from the above procedure is close to the amount of vitamin added, indicating vitamin E was successfully embedded into the fibers.

TABLE 1

| | CHAAE5 | CHAAE10 | CHAAE20 |
|---|---|---|---|
| Vitamin E added % | 5 | 10 | 20 |
| Vitamin E determined % | 3.71 | 9.97 | 19.06 |

Nuclear Magnetic Resonance

[Emim][OAc]: δ ppm (360 MHz, dmso) 9.73-9.81 (m, 1H), 7.72 (d, 1H, J=1.6 Hz), 7.81 (s, 1H) 4.19 (q, 3H, J=7.3 Hz), 3.84 (s, 3H), 1.39 (t, 5H, J=7.3 Hz).

Vitamin E: ¹H NMR (360 MHz, dmso-d₆) δ ppm 7.38 (s, 1H), 3.35 (s, 1H), 2.07-1.96 (m, 9H), 1.79-1.66 (m, 2H), 1.59-0.97 (m, 23H), 0.86 (d, J=6.67 Hz, 6H), 0.83 (d, J=6.62 Hz, 6H).

The NMR showed that there is no residual IL left in the fibers. In ¹H NMR spectra of all of the fibers-chitin (CH), chitinous-alginate (CHA), and chitinous-alginate-Vitamin E (CHAE5, CHAE10 and CHAE20), no [Emim][OAc] signals were detected, indicating that the IL was completely removed from the fibers. (This was also proved by two other analyses presented below, FT-IR spectroscopy that showed no IL-related peaks present and intracutenous irritation test that indicated absence of toxic leachable components in fibers' extracts, and thus no irritation effect.) The NMR data also shows that Vitamin E-loaded fibers contained the Vitamin E: Signals in ¹H NMR spectrum of the chitin-alginate-Vitamin E fibers (CHAE5, CHAE10 and CHAE20) matched the signals associated with Vitamin E, proving the presence of Vitamin. E in the fibers. NMR technique was not applicable for quantitative analysis of other fiber components.

Fourier Transform Infrared Spectroscopy, FT-IR.

[Emim][OAc, cm⁻¹]: 3362, 3073, 2981, 1562, 1451, 1427, 1384, 1331, 1172, 907, 759, 701, 667; Chitin, cm⁻¹: 3445, 3252, 3099, 2918, 2855, 1660, 1620, 1541, 1461, 1422, 1376, 1308, 1257, 1201, 1150, 1110, 1065; Vit E, cm⁻¹: 3462, 2950, 2929, 2860, 2840, 1456, 1421, 1378, 1340, 1260, 1245, 1208, 1156, 1110, 1072; Calcium Alginate, cm⁻¹: 3585, 2950, 2900, 1597, 1404, 1268, 1100, 1030.

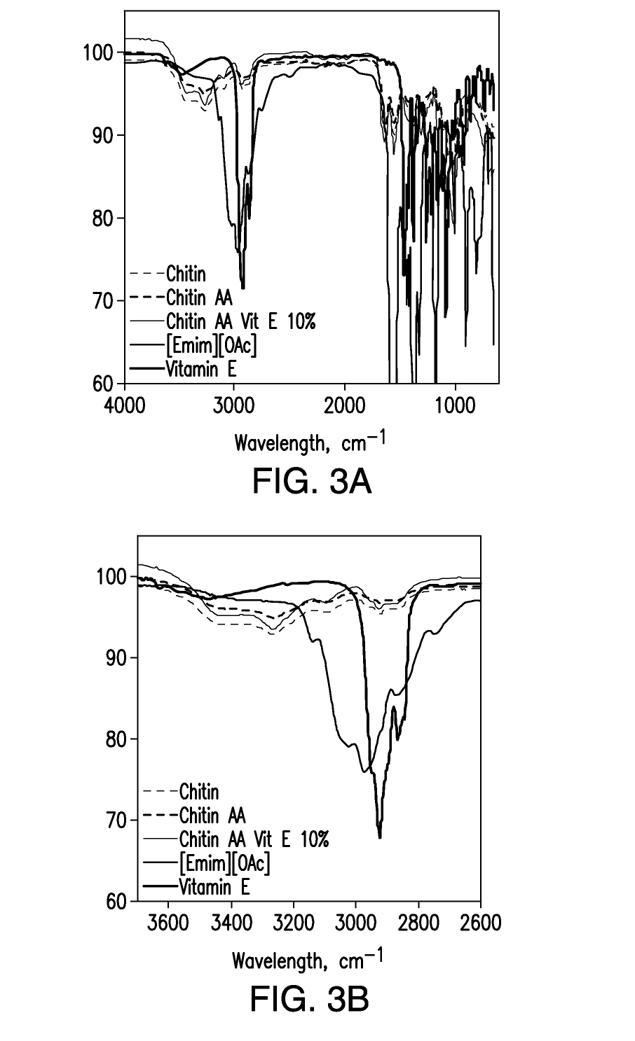

Compositional analysis confirmed the presence of both chitin and alginate in the fibers: Structural determination of fibers composition through FT-IR spectroscopy allowed for the determination of a series of narrow absorption bands, typical for polysaccharides, chitin and alginate (note that some alginic acid was also present.) FT-IRs of composite fibers vs. FT-IRs of fibers components are presented in FIG. 3A. A portion of FIG. 3A is enlarged in FIG. 3B to reveal peaks from vitamin E. FT-IR thus confirmed the presence of vitamin E in the vitamin-loaded fibers CHAE5, CHAE10 and CHAE20. These results are in agreement with NMR results discussed above. No [Emim][OAc] was found in the fibers, as shown in FIG. 3A. Results are in agreement with results of NMR analysis above, and intracutenous irritation test below demonstrating no toxic leachable compounds.

Thermal Stability of Fibers.

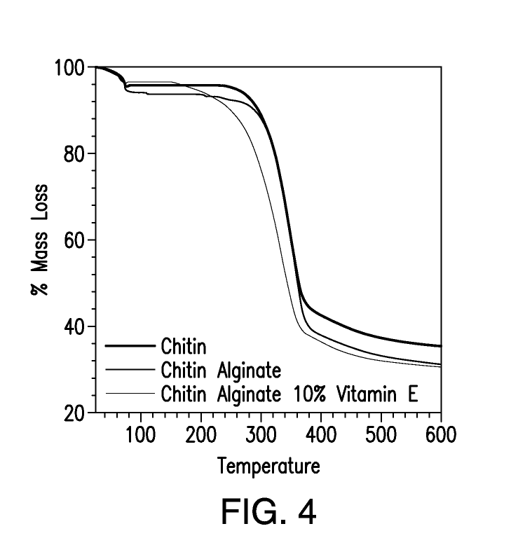

Thermal stability of fibers are indicated by shelf-life/temperature stability of the fibers until the time of sale or use. The thermal stability of fibers therefore is important to determine guidelines for expected handling and temperature exposure. Decomposition temperatures (T_{5% decomposition}) were determined using thermal gravimetric analysis (TGA) and the results are illustrated in FIG. 4. As shown in FIG. 4, fibers appeared to be thermally stable to the temperatures of c.a. 190-267° C. Fibers composed of chitin and chitinous-alginate are more thermally stable than those loaded with vitamin E. It seems that Vitamin E present in fibers decreases the decomposition temperature of the fibers when compared to chitin-alginate composite fibers without such additive.

Water content of the fibers were also estimated from the TGA plot in FIG. 4 based on the onset temperature (T_{5% onset}) and the results listed in Table 2.

TABLE 2

| | T_{5% onset} ° C. | % water |
|---|---|---|
| CH | 267 | 4.25 |
| CHA | 236.3 | 5.85 |
| CHAE10 | 190.5 | 3.33 |

As shown in Table 2, alginate-containing fibers contain more water than neat chitin fibers. the water content values of the fibers vary from c.a. 3 to 5%, which is well below the standard specification moisture content for chitosan fiber (15-20%) and alginate fibers (15-25%) reported in Standard Specifications for Alginate Fibers (ASTM F2064) and Chitin Fibers (for medicinal use).

Surface Characterization with Optical Microscopy

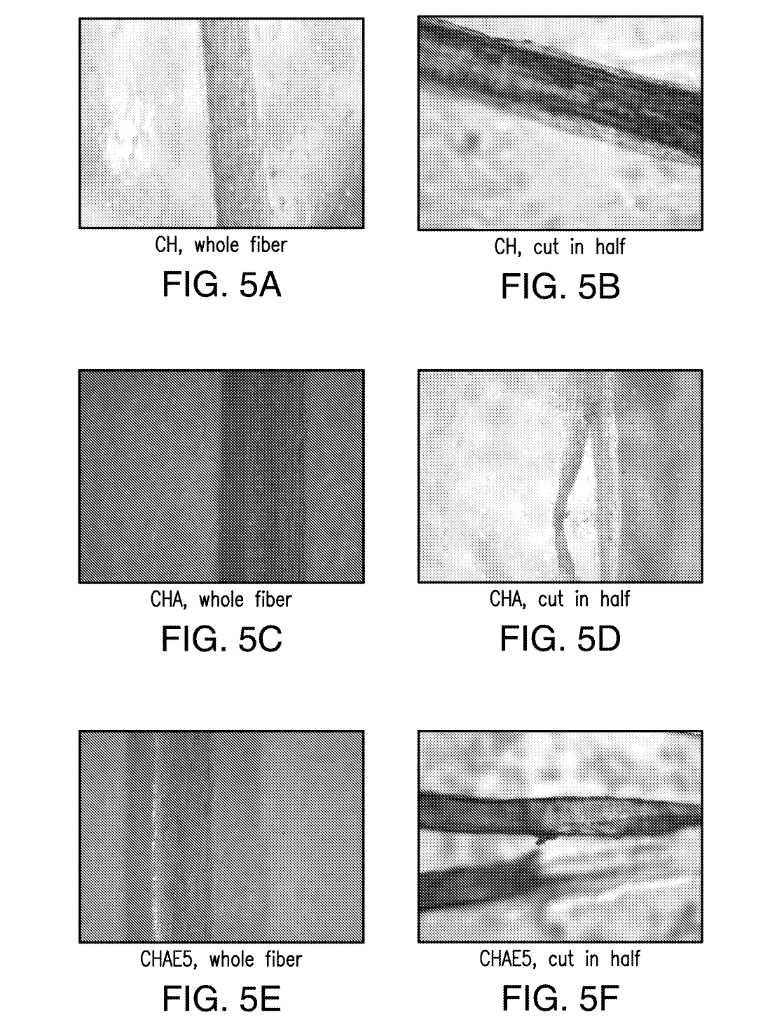

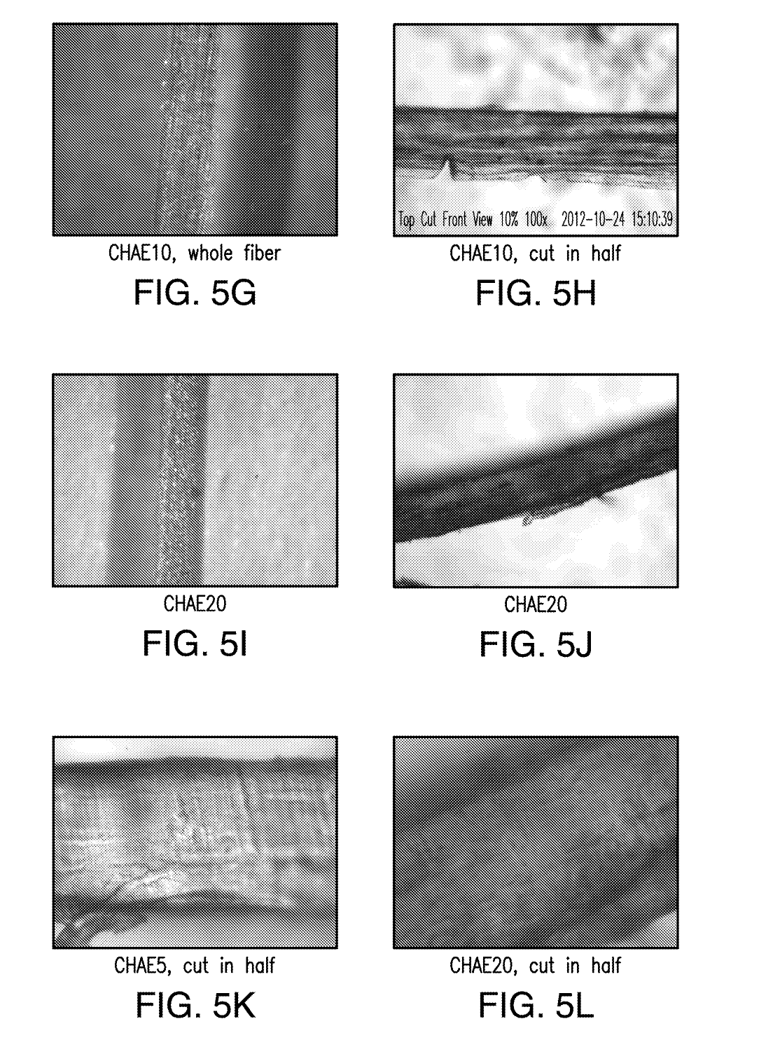

Surface characterization of the fibers disclosed herein has shown the surface of the fibers are uniform and homogeneous. Optical microscopy was used to magnify the fiber images so that specimen details can be observed to provide insight into the macrostructure of fibers. Optical microscopy shown in FIGS. 5A-L revealed no substantial differences in CH, CHA or CHAE fibers. The microscopies shown in FIGS. 5A-J were taken at 100 times magnification while the FIG. 5K and FIG. 5L were taken at 400 times magnification. There was no entrapped air in the composite fibers and no voids (dark areas) observed indicating that at the surface the fibers were uniform and homogeneous. For fibers loaded with Vitamin E, a higher magnification was used to see if there is any correlation between the amount of vitamin E loaded and the alignment of the biopolymers within the fibers' structure. No visual difference between fibers with 5 and 10% of Vitamin E was observed, in FIG. 5K and FIG. 5L.

Surface Characterization with Scanning Electron Microscopy (SEM)

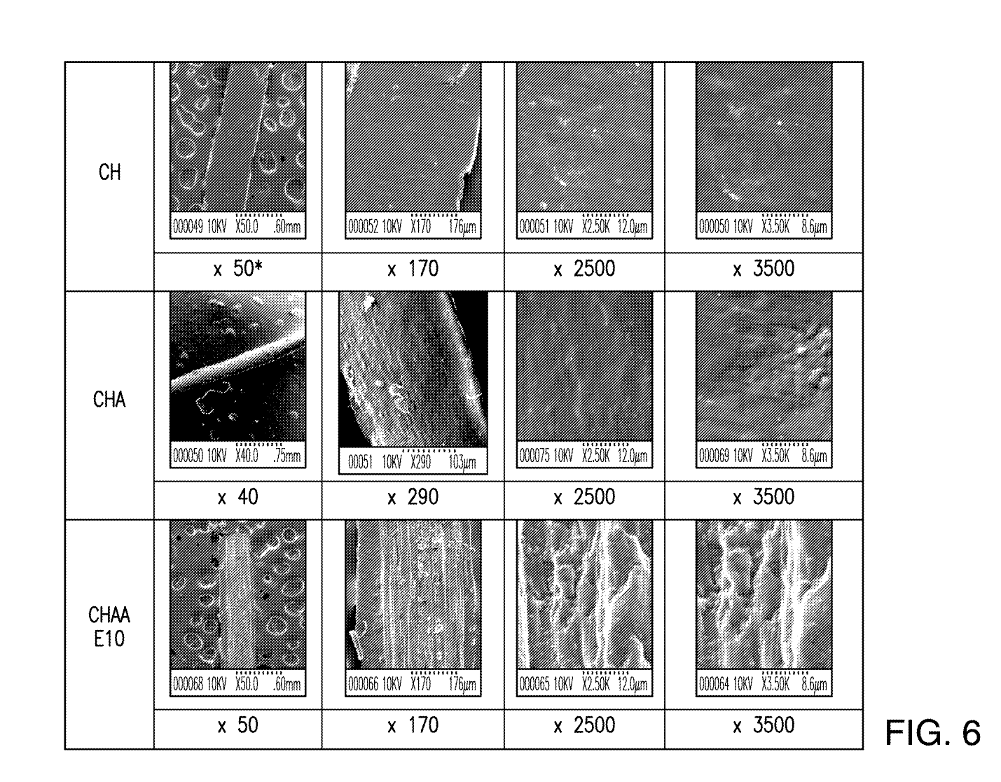

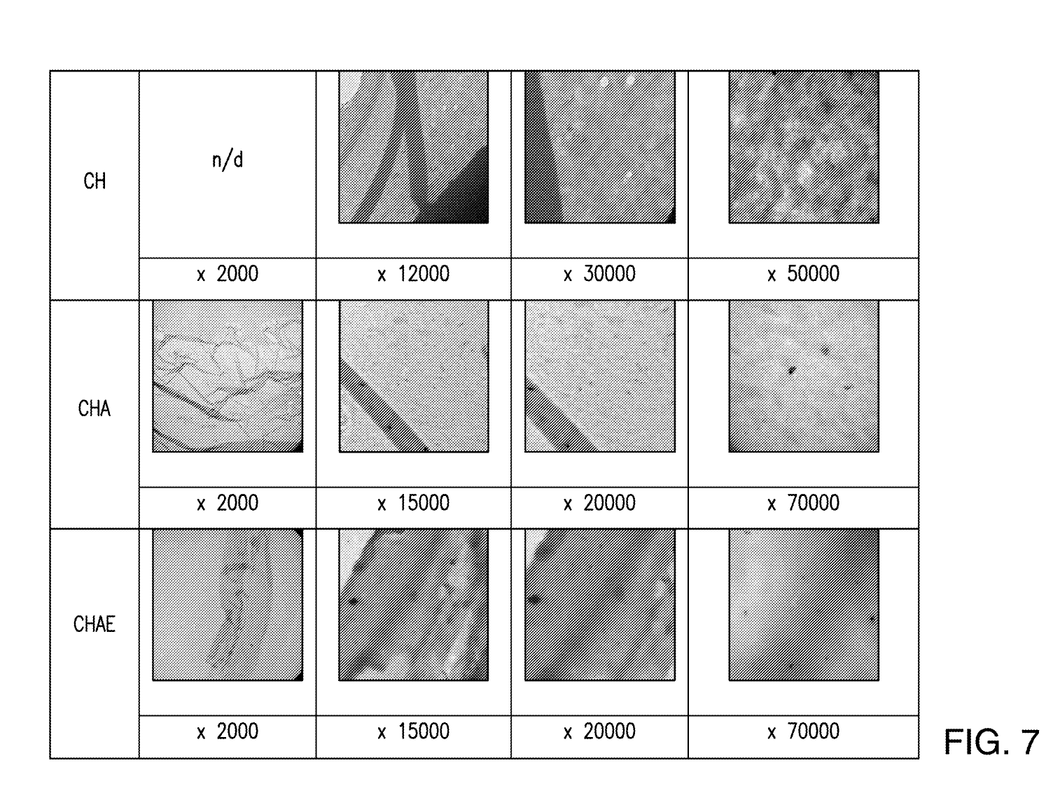

Scanning electron microscopy provided greater magnification of the fibers surface to observe specimen details in order to provide insight into the macrostructure of fibers. FIG. 6 shows the SEM micrographs of the surfaces of the fibers CH, CHA, and CHAAE10. The XNo. below each SEM micrograph indicates the magnification used. All fibers appeared to be continuous, with aligned fiber orientation. Similarly to the fibers made from pure chitin, or chitin/alginate, the surface of the fibers with embedded Vitamin E exhibits a homogeneous morphology and no obvious splits. However, the surface of Vitamin-loaded fibers is not very smooth, indicating that vitamin E is present not only inside the fiber but might be also present on the fiber surface.

Morphological Study Using Transmission Electron Microscopy (TEM)

TEM, or morphology studies were performed on the fibers and the results shown in FIG. 7. The XNo. below each TEM micrograph indicates the magnification used. The TEM demonstrated that produced chitin, chitinous-alginate and Vitamin E-loaded fibers exhibited a homogeneous morphology, indicating blend homogeneity between all three components. Vitamin E and alginic acid/alginate are evenly distributed within the fibers. This is proved by the absence of higher-density/dark regions around the edges. Darker lines on x 2000 magnification image are possibly due to the sliding of hydrogen-bonded sheets of chains as force-induced transitions in biopolymer molecules due to pulling.

Technical Characterization: Diameter of the Fibers in mm

The diameter of fibers is a very important factor and affects several technical parameters, such as denier and elongation. Ten measurements at different points of each fiber were recorded and the average diameter was determined. Results are reproducible and controllable. The fibers' diameter strongly depends on the manufacturing process, and all of fibers were hand-pulled using the procedure illustrated in FIG. 1. The diameters data are presented in Table 3.

TABLE 3

| Fiber | # | | Trial 1 | Trial 2 | Trial 3 | Trial 4 | Trial 5 | Avg. |
|---|---|---|---|---|---|---|---|---|
| Chitin | 1 | mm | 0.089 | 0.10 | 0.10 | 0.10 | 0.089 | 0.097(6) |
| Fiber | | inches | 0.0035 | 0.0040 | 0.0040 | 0.0040 | 0.0035 | 0.0038(2) |
| | 2 | mm | 0.089 | 0.076 | 0.076 | 0.064 | 0.089 | 0.079(9) |
| | | inches | 0.0035 | 0.0030 | 0.0030 | 0.0025 | 0.0035 | 0.0031(3) |
| | 3 | mm | 0.064 | 0.064 | 0.076 | 0.089 | 0.064 | 0.071(10) |
| | | inches | 0.0025 | 0.0025 | 0.0030 | 0.0035 | 0.0025 | 0.0028(4) |
| | 4 | mm | 0.064 | 0.089 | 0.102 | 0.089 | 0.089 | 0.086(12) |
| | | inches | 0.0025 | 0.0035 | 0.0040 | 0.0035 | 0.0035 | 0.0034(5) |
| | 5 | mm | 0.051 | 0.064 | 0.076 | 0.076 | 0.076 | 0.069(10) |
| | | inches | 0.0020 | 0.0025 | 0.0030 | 0.0030 | 0.0030 | 0.0027(4) |
| Chitin- | 1 | mm | 0.076 | 0.064 | 0.089 | 0.076 | 0.102 | 0.081(13) |
| calcium | | inches | 0.0030 | 0.0025 | 0.0035 | 0.0030 | 0.0040 | 0.0032(5) |
| alginate | 2 | mm | 0.10 | 0.089 | 0.11 | 0.089 | 0.10 | 0.099(9) |
| fiber | | inches | 0.0040 | 0.0035 | 0.0045 | 0.0035 | 0.0040 | 0.0039(3) |
| | 3 | mm | 0.089 | 0.10 | 0.089 | 0.11 | 0.10 | 0.099(9) |
| | | inches | 0.0035 | 0.0040 | 0.0035 | 0.0045 | 0.0040 | 0.0039(3) |
| | 4 | mm | 0.089 | 0.076 | 0.089 | 0.10 | 0.076 | 0.086(9) |
| | | inches | 0.0035 | 0.0030 | 0.0035 | 0.0040 | 0.0030 | 0.0034(4) |
| | 5 | mm | 0.076 | 0.089 | 0.10 | 0.089 | 0.10 | 0.091(9) |
| | | inches | 0.0030 | 0.0035 | 0.0040 | 0.0035 | 0.0040 | 0.0036(4) |
| Chitin- | 1 | mm | 0.16 | 0.12 | 0.13 | 0.14 | 0.14 | 0.138(14) |
| calcium | | inches | 0.0062 | 0.0047 | 0.0051 | 0.0055 | 0.0055 | 0.005(0) |
| alginate | 2 | mm | 0.10 | 0.16 | 0.27 | 0.14 | 0.34 | 0.202(99) |
| 5% | | inches | 0.0039 | 0.0062 | 0.011 | 0.0055 | 0.013 | 0.008(4) |
| vitamin | 3 | mm | 0.24 | 0.09 | 0.09 | 0.26 | 0.11 | 0.158(84) |
| E fiber | | inches | 0.0094 | 0.0035 | 0.0035 | 0.010 | 0.0043 | 0.0062(3) |
| | 4 | mm | 0.18 | 0.31 | 0.23 | 0.12 | 0.11 | 0.19(82) |
| | | inches | 0.0073 | 0.012 | 0.0094 | 0.0049 | 0.0045 | 0.0077(3) |
| | 5 | mm | 0.15 | 0.28 | 0.17 | 0.39 | 0.14 | 0.226(107) |
| | | inches | 0.0059 | 0.011 | 0.0066 | 0.015 | 0.0055 | 0.0088(4) |
| Chitin- | 1 | mm | 0.09 | 0.12 | 0.1 | 0.11 | 0.18 | 0.12(35) |
| calcium | | inches | 0.0035 | 0.0047 | 0.0039 | 0.0043 | 0.0070 | 0.0047(1) |
| alginate | 2 | mm | 0.09 | 0.08 | 0.08 | 0.09 | 0.10 | 0.0088(8) |
| 10% | | inches | 0.0035 | 0.0031 | 0.0031 | 0.0035 | 0.0039 | 0.0034(0) |
| vitamin | 3 | mm | 0.09 | 0.17 | 0.13 | 0.10 | 0.12 | 0.122(31) |
| E fiber | | inches | 0.0059 | 0.0055 | 0.0051 | 0.0055 | 0.0055 | 0.0048(1) |
| | 4 | mm | 0.10 | 0.12 | 0.11 | 0.11 | 0.16 | 0.12(23) |
| | | inches | 0.0039 | 0.0047 | 0.0043 | 0.0043 | 0.0062 | 0.0047(1) |
| | 5 | mm | 0.15 | 0.14 | 0.13 | 0.14 | 0.14 | 0.14(7) |
| | | inches | 0.0059 | 0.0055 | 0.0051 | 0.0055 | 0.0055 | 0.0055(0) |
| Chitin- | 1 | mm | 0.15 | 0.18 | 0.12 | 0.12 | 0.16 | 0.146(26) |
| calcium | | inches | 0.0059 | 0.0070 | 0.0047 | 0.0047 | 0.0063 | 0.0057(1) |
| alginate | 2 | mm | 0.12 | 0.11 | 0.19 | 0.13 | 0.12 | 0.134(32) |
| 20% | | inches | 0.0047 | 0.0043 | 0.0074 | 0.0051 | 0.0047 | 0.0052(1) |
| vitamin | 3 | mm | 0.09 | 0.15 | 0.14 | 0.12 | 0.12 | 0.124(23) |
| E fiber | | inches | 0.0035 | 0.0059 | 0.0055 | 0.0047 | 0.0047 | 0.0048(1) |
| | 4 | mm | 0.14 | 0.16 | 0.13 | 0.15 | 0.16 | 0.148(13) |
| | | inches | 0.0055 | 0.0063 | 0.0051 | 0.0059 | 0.0063 | 0.0058(1) |
| | 5 | mm | 0.19 | 0.18 | 0.15 | 0.14 | 0.15 | 0.162(21) |
| | | inches | 0.0074 | 0.0070 | 0.0059 | 0.0055 | 0.0059 | 0.0063(1) |

Average for all trials

| | | |
|---|---|---|
| Chitin Fibers | mm | 0.080(1) |
| | inches | 0.0031(4) |
| Chitin-calcium alginate | mm | 0.091(7) |
| Fibers | inches | 0.0036(5) |
| Chitin-calcium alginate | mm | 0.182(35) |
| 5% vitaminE fiber | inches | 000072(1) |
| Chitin-calcium alginate | mm | 0.118(18) |
| 10% vitaminE fiber | inches | 0.0046(1) |
| Chitin-calcium alginate | mm | 0.142(14) |
| 20% vitaminE fiber | inches | 0.0056(1) |

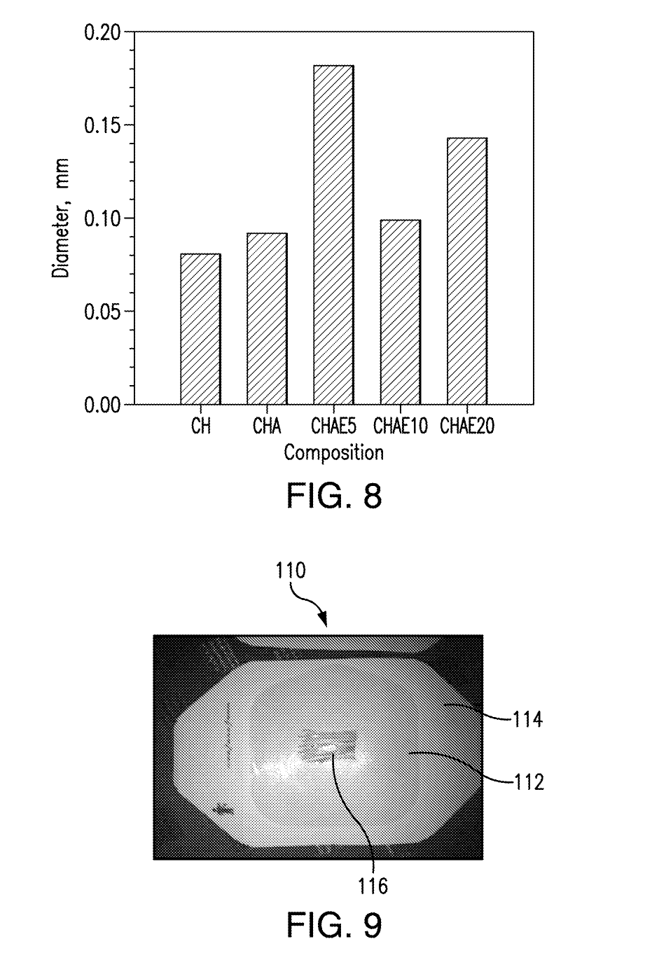

The average diameter of each fiber type were further compared in FIG. 8, While chitinous-alginate fibers with or without Vitamin E are slightly thicker than the pure chitin fibers, the diameters of the fibers with embedded Vitamin E vary.

Denier Determination

ASTM protocol D1577 was followed to determine the denier of linear density of the fibers and the results listed in Table 4 below. All the novel fibers are much denser than fibers prepared from chitosan or alginate. Denier or den is a unit of measure for the linear mass density of fibers, which is the mass of fibers in grams per 9,000 meters. The denier is based on a natural standard, i.e., a single strand of silk is approximately one denier. For instance, standard specifications for the Denier is 2-3 for the alginate and 1.5-3 for chitosan, (Standard Test Methods for Linear Density of Textile Fibers. Active Standard, ASTM D1577-07, 2007), while the fibers disclosed herein are two orders of magnitude denser. Denier is proportional to the diameter and also strongly depends on the manufacturing process.

TABLE 4

| | | | | | | | | | D avg. |
|---|---|---|---|---|---|---|---|---|---|
| | | | | | | | | | highest/ |
| | | | | | | | Linear | D | lowest |
| | | | | | | | density | average | dropped |
| | Diameter | | Length | | Weight | | grams | Grams/ | Grams per |
| | mm | In | mm | M | mg | g | per 9,000 m | 9,000 m | 9,000 meters |
| CH | 0.074 | 0.0019 | 30 | 0.03 | 1.50 | 0.0015 | 450.00 | 401(103)^{a} | 389(83)^{a} |
| | 0.074 | 0.0019 | 30 | 0.03 | 1.40 | 0.0014 | 420.00 | | |
| | 0.074 | 0.0019 | 30 | 0.03 | 1.10 | 0.0011 | 330.00 | | |
| CHA | 0.088 | 0.00347 | 30 | 0.03 | 1.70 | 0.0017 | 510.00 | 339(87)^{a} | 330(67)^{a} |
| | 0.088 | 0.00347 | 30 | 0.03 | 1.00 | 0.001 | 300.00 | | |
| | 0.088 | 0.00347 | 30 | 0.03 | 1.20 | 0.0012 | 360.00 | | |
| CHAE5 | 0.19 | 0.0775 | 30 | 0.03 | 1.50 | 0.0015 | 450.00 | 382(117)^{a} | 390(78)^{a} |
| | 0.19 | 0.0775 | 30 | 0.03 | 1.10 | 0.0011 | 330.00 | | |
| | 0.19 | 0.0775 | 30 | 0.03 | 1.80 | 0.0018 | 540.00 | | |
| CHAE10 | 0.088 | 0.0035 | 30 | 0.03 | 0.50 | 0.0005 | 150.00 | 219(80)^{a} | 210(68)^{a} |
| | 0.088 | 0.0035 | 30 | 0.03 | 1.00 | 0.001 | 300.00 | | |
| | 0.088 | 0.0035 | 30 | 0.03 | 0.60 | 0.0006 | 180.00 | | |
| CHAE20 | 0.162 | 0.0064 | 30 | 0.03 | 1.50 | 0.0015 | 450.00 | 487(74)^{a} | 485(58)^{a} |
| | 0.162 | 0.0064 | 30 | 0.03 | 1.80 | 0.0018 | 540.00 | | |
| | 0.162 | 0.0064 | 30 | 0.03 | 1.60 | 0.0016 | 480.00 | | |

^{a}Measurements were taken on 10 samples for each fiber type following ASTM protocol D1577 for determination of fiber's linear density. Only data from three samples are shown.

Water Uptake/Absorption Capacity Determination

Standard protocol from British Pharmacopoeia Monograph for Alginate Dressings and Packings (British Pharmacopoeia Monograph for Alginate Dressings and Packings, 1994) were used to determine the water uptake/absorption capacity of the fibers and the results listed in Table 5 below. The absorption capacity was measured according to the standard protocol from British Pharmacopoeia monograph for Alginate Dressings and Packings (British Pharmacopoeia Monograph for Alginate Dressings and Packings, 1994). A dry piece of fiber was placed in a screw top vial in 20 mL of deionized water. The fiber piece was immersed in water for 24-48 hours at 25° C. The sample was lifted from one end and hold in air for 30 s, then placed on Kimwipe and covered with another sheet of Kimwipe before the weight was measured using a microbalance. The experiment was continued for 48 hours, i.e., until the samples reached the equilibrium. The swelling degree was calculated according to the following equation:

% absorption capacity={(wet weight−dry weight)/dry weight}×100

TABLE 5

| | Mass increase | Mass increase |
|---|---|---|
| | after 24 Hours | after 40 Hours |
| Composition | g_{wet}/g_{dry}^{a} | g_{wet}/g_{dry}^{a} |
| CH | 2.57 | 2.63 |
| CHA | 2.06 | 2.24 |
| CHAE5 | 2.75 | 2.90 |
| CHAE10 | 2.38 | 2.38 |
| CHAE20 | 2.58 | 2.60 |

^{a}Average from three trials.

As shown in Table 5, the absorption capacity of the fibers disclosed herein falls within the required limits for fibers used in wound dressings. The water absorption capability is an important attribute for wound dressings, since wounds can release large amount of exudates (at a rate of 20 mg/cm²/h), and wound dressing has to be able to keep wound moist but not wet. Absorption capacity values for all the fibers were consistent with values reported for chitosan and alginate. Chitin and chitinous-alginate fibers absorbed most of the water during the first 24 hours. Thus, after 40 hours, the fibers absorb only 10-15% more water than after 24 hours. The situation is slightly different when Vitamin E was used as an additive. Fibers with added Vitamin E, absorbed all the water after the first 24 hours. There is no difference in water uptake after 24 or 40 hours. Literature values for water uptake are 2.6-6.1 g/g for alginate and 2-6 g/g for chitosan fibers as reported in British Pharmacopoeia Monograph for Alginate Dressings and Packings, 1994. The values for the composite fibers are between 2-3 g/g for all tested materials.

Tensile Strength/Elongation of the Fibers

Depending on the fibers' use and method of production (woven, knitted, non-woven), the fibers have to satisfy desired mechanical properties requirement, such as tensile testing. Tensile strength, or the fiber's capacity to be stretched without breaking, gives the information on how much tension fibers can endure. Many of the values depend on the manufacturing process and purity and composition of the fibers. ASTM D3822-07, Standard Test Method for Tensile Properties of Single Textile Fibers were used to test the tensile strength/elongation of the fibers and the results are shown in Table 6.

TABLE 6

| | | | Peak | Peak | Peak | Break | Break | Yield | Yield |
|---|---|---|---|---|---|---|---|---|---|
| Sample | Width | Thickness | Load | Stress | Stress | Stress | Elong. | Stress | Elong. |
| I.D. | In | In | Lb | PSI | MPa | PSI | % | PSI | % |
| CH | 0.003 | 0.066 | 0.26 | 32350 | 216.2 | 32350 | 5.0 | 30980 | 4.55 |
| CHA | 0.004 | 0.066 | 0.35 | 31621 | 211.5 | 31621 | 5.4 | 30134 | 4.9 |
| CHAE10 | 0.005 | 0.066 | 0.45 | 34706 | 216.7 | 34706 | 6.3 | 33671 | 5.7 |

TABLE 7

| Fibers | Tensile/Elongation | Reference |
|---|---|---|
| Chitosan fibers | 133.65 MPa/11.2% | Materials Letters 2012, 84, 73-76 |
| Chitosan fibers from | 162.58 MPa/12.9% | Materials Letters 2012, 84, 73-76 |
| LiOH/urea | | |
| Chitosan fibers produced | 204.9 MPa/not provided | Micromol. Biosci. 2004, 4, 811-819 |
| with purpose of strength | | |
| enhancement | | |
| Sodium Alginate | 1.21 cN/dtex ^{a} or 193.6 MPa/8.3% | J. Eng. Fibers Fabrics 2011, 6, 69. |
| Chitosan-Alginate Blend | 12.25 cN/tex ^{a} or 15.9 MPa - | J. Macromolecul. Sci., Part A: Pure |
| fibers | low as wet fiber used/23.3% | Appl. Chem., 2005, 4, 723-732 |
| Chitin Cellulose Blend | 256 MPa/8.7% | Cellulose Chem. Tech. 2009, 43 393. |
| Chitosan - octa ammonium | 80 MPa | Carbohydrate Polym. 2011, 86, 1151. |
| (OA) silsesquioxane | | |
| Chitosan-poly(ε- | 0.184-1.49 MPa/1.74-25.88% | AAPS PharmSciTech 2007, 8, E1-E11 |
| caprolactone), Different ratio | | |
| Cellulose-Alginate Blend | 1.57 cN/dtex/10.8% | J. Eng. Fibers Fabrics 2011, 6, 69 |

^{a} Term “tex” is similar to denier and is defined as the weight in grams of 1000 meters of the material. The term is used outside the USA. A related term is decitex (dtex, 0.1 tex). 1 cN/tex = (10 × d) MPa and 1_cN/dtex = (100 × d) MPa, where d is the specific gravity of the fiber. Specific gravity for chitin is d_{CH} = 1.2 and for alginate equal d_{CaAlg} = 1.6, respectively. For composite fibers, d_{CHA} = χ_{CH} × d_{CH} + χ_{CaAlg} × d_{CaAlginate} = 0.75 × 1.2 + 0.25 × 1.6 = 1.3, where χ is mass fraction.

All of biocomposite fibers have tensile strength values between 211 and 217 MPa with 5-6% elongation. Based on the values listed in Table 7, which are reported for chitosan and alginate used in the medical field, the data in Table 6 suggested that the fibers exceed the tensile specifications needed for medical applications. The biocomposite fibers therefore have the required strength for a wound dressing material.

Additive Leaching Studies

Vitamin E (α-tocopherol) is a fat soluble vitamin, and is not soluble in water or standard buffer solutions owing to the hydrophobic repulsion between water and vitamin molecules. The solubility values are 20.9×10⁻⁶ mass fraction (g vitamin E/g water) or 0.87×10⁻⁶ mol fraction (mole vitamin E/mole water) according to Dubbs, M. D. and Gupta, R. B. “Solubility of vitamin E (α-Tocopherol) and Vitamin K₃ (Menadione) in ethanol-water mixtures”. J. Chem. Eng. Data 1998, 43, 590-591. Due to the solubility limits in buffer solutions, four different types of releasing media were prepared according to Taepaiboon et al. “Vitamin-loaded electrospun cellulose acetate nanofiber mats as transdermal and dermal therapeutic agents of vitamin A acid and vitamin E”. Eur. J. Pharm. Biopharm. 2007, 67, 387-397. The first releasing medium was prepared by adding 0.5% (v/v) of a non-ionic surfactant, polysorbate 80 (hereafter, Tween 80), to the pH 4.5 and pH 5.2 buffer solutions to help solubilize the vitamin E from the vitamin-loaded samples. These media are hereinafter referred to as B/T. The other releasing media were prepared by adding 0.5% (v/v) of Tween 80 and 10% (v/v) of methanol to the buffer solution. These media are hereinafter referred to as B/T/M.

Procedure for the construction of calibration curve: Vitamin E was weighed and transferred to volumetric flasks, diluted to volume with the required buffer, vortexed for 5 min, sonicated for 30 min, and left for 12 hours at room temperature to reach the equilibrium. Then the solution was filtered with syringe filter tip to obtain clear stock solution. Then 5-8 various dilutions were made by the addition of fresh buffer. Selected dilutions were analyzed by UV-Vis and absorbance at 292 nm was selected for analysis. The amount of vitamin E in the buffer solution was determined using UV-Vis against the predetermined calibration curve for each buffer. These data were carefully calculated to determine the cumulative amount of vitamin E released from the samples at 24 hours immersion period and the results are listed in Table 8 below.

The results show that vitamin E has very low solubility in these buffers, and Vitamin E leaches until it reaches maximum solubility, therefore resulting in approximately the same amount released independently of loaded Vitamin E amount. The more acidic media can accommodate larger amount of Vitamin E. The test results indicated that vitamin E can be distributed throughout the composite fibers, allowing for controlled release and thus provide improved wound healing.

TABLE 8

| | | | Vitamin E | | VitE | |
|---|---|---|---|---|---|---|
| | Fiber | Buffer | embedded, | Absorb. | released | |
| | mass, mg | mass, g | mg | UV-Vis | mg/mL | Calibration curve |
| 4.5 B/T | | | | | Avg = 0.034 | 295 nm |
| CHAE5 | 11.18 | 3.37 | 0.559 | 0.072 | 0.0294 | Coefficients: |
| CHAE10 | 8.41 | 3.20 | 0.841 | 0.080 | 0.0320 | b[0] 0.014447619 |
| CHAE20 | 12.31 | 3.17 | 2.466 | 0.110 | 0.0422 | b[1] 2.939507389 |
| | | | | | | r² 0.994607265 |
| 4.5 B/T/M | | | | | Avg = 0.019 | 295 nm |
| CHAE5 | 10.11 | 3.427 | 0.5055 | 0.110 | 0.0196 | Coefficients: |
| CHAE10 | 10.56 | 3.643 | 1.056 | 0.101 | 0.0180 | b[0] −2.49E−03 |
| CHAE20 | 9.9 | 3.27 | 1.98 | 0.116 | 0.0206 | b[1] 5.73274663 |
| | | | | | | r² 0.999928874 |
| 5.2 B/T | | | | | Avg = 0.011 | 304 nm |
| CHAE5 | 9.03 | 3.16 | 0.4515 | 0.061 | 0.0140 | Coefficients: |
| CHAE10 | 9.59 | 3.572 | 0.959 | 0.036 | 0.0070 | b[0] 0.011348789 |
| CHAE20 | 10.48 | 3.072 | 2.096 | 0.052 | 0.0115 | b[1] 3.560159139 |
| | | | | | | r² 0.993585225 |
| 5.2 B/T/M | | | | | Avg = 0.007 | 295 nm |
| CHAE5 | 9.74 | 3.248 | 0.487 | 0.034 | 0.0055 | Coefficients: |
| CHAE10 | 9.28 | 3.601 | 0.928 | 0.055 | 0.0080 | b[0] 8.60E−03 |
| CHAE20 | 11.41 | 3.11 | 2.282 | 0.042 | 0.0072 | b[1] 4.671509662 |
| | | | | | | r² 0.998778267 |

The tensile strengths of chitin fiber, chitinous-alginate fiber, chitinous-alginate-vitamin E fiber were tested along with commercially available alginate fiber and chitosan fibers using an MTS Q-Test 25 machine with a specially designed pneumatic grip suitable for thin and flexible fiber testing. Fibers of uniform cross-section from each type were tested using a load cell of 22.4 Newton capacity (5 lbs) and a cross-head speed maintained at 1.27 mm min⁻¹. The results are summarized in Table 9 below. The results indicate that prepared composite fibers were comparable to the pure chitin fibers (CH) and stronger than either alginate or chitosan fibers. The elongation of the composite fibers was determined to be 5-6%. The composite fibers were comparable to the pure chitin fibers and substantially denser when compared to reported values for either chitosan or alginate fibers based on the calculations of denier, linear mass density of fibers. All fibers were thermally stable to the temperatures of 190-267° C. The moisture content of the composite fibers was lower than those reported for chitosan and alginate, while moisture absorbance values were consistent with the reported values.

TABLE 9

| | Algi- | | Chi- | Chitin/ | Chitin/alginate/ |
|---|---|---|---|---|---|
| | nate | Chitosan | tin | alginate | 10% Vit E |
| Property | Fibers | Fibers | fibers | fibers | fibers |
| Denier* g/m | 2-3 | 1.5-3 | 389 | 330 | 210 |
| Tenacity** MPa | 194 | 134 | 216 | 212 | 217 |
| Elongation, % | 12-20 | 10-20 | 5 | 5.4 | 6.3 |
| Moisture content | 15-25 | 15-20 | 3-5 | 3-5 | 3-5 |
| Gel swelling in | 2.6-6.1 | 2-6 | 2.6 | 2.2 | 2.4 |
| water, g/g | | | | | |

*linear mass-density of textile fiber calculated as one gram per 9000 meters
**(property of fibers which keeps them from parting without considerable force

Additionally, the morphologies of the chitinous-alginate composite fibers were analyzed. Both surface and morphology studies demonstrated that the chitin, chitinous-alginate and Vitamin E-loaded fibers exhibited a homogeneous morphology, indicating blend homogeneity between all three components. All produced fibers were continuous, with aligned fiber orientation.

### Example 3

### Wound Dressing Preparation and Evaluation

The biocompatibility/efficacy testing included a cytotoxicity test (MEM Elution Using L-929 Mouse Fibroblast Cells (ISO), an irritation/intracutaneous reactivity test using Intracutaneous Irritation Test (ISO) protocol, and a preliminary efficacy tests using a rat wound healing model.

Cytotoxicity Test

The cytotoxicity test was performed in accordance with the requirements specified in ISO 10993-5; 2009. None of the tested fibers produced an increase in cell growth of L929 mouse fibroblasts during the 72 h duration of the test and therefore did not pass the test under the test conditions employed. The results are not unusual for materials containing chitin. Literature reports also show suppression in cell proliferation when using cell line L-929 and E-MEM assay in cytotoxicity studies of chitin and chitin derivatives, Mori et al. “Effects of chitin and its derivatives on the proliferation and cytokine production of fibroblasts in vitro”. Biomaterials 1997, 18, 947-951. It was also noted that although cell suppression was observed during the first six days, the cultures recovered in cell growth on day 9. This behavior can be attributed to the interaction of the chitin with growth factors, thus immobilizing them as reported in Khor E. and Lim, L. Y. “Implantable applications of chitin and chitosan”. Biomaterials 2003, 24, 2339-2349, as well as to the cell type used. In cell cultures, only a few of the in vivo variables are accounted for, therefore in vitro cytotoxicity test cannot be viewed as a simulation of the in vivo results, according to Rodrigues et al “Biocompatibility of Chitosan Carriers with Application in Drug Delivery”. J. Funct. Biomater. 2012, 3, 615-641.

Irritation/Intracutaneous Reactivity Testing

The requirements of the ISO Intracutaneous Reactivity Test have been met by all tested fibers. The study was conducted in accordance with ISO 10993-10: 2010 Standard, Biological Evaluation of Medical Devices, Part 10: Tests for Irritation and Skin Sensitization, Pages 11-14.

The purpose of this test was to determine if any chemicals that may leach or be extracted from the test articles were capable of causing local irritation in the dermal tissues of rabbits. The irritation reaction of the test extracts were compared to vehicle controls and recorded over a 72-hour period. At the beginning and at the end of the extraction, the solutions appeared clear and free of particulates and all the test articles were intact with no macroscopically observable degradation. After intracutaneous administration of the extraction mixtures, the animals were observed daily and none of them showed abnormal clinical signs during the 72 hour test period. Also, there were no significant dermal reactions observed at the injected sites on the rabbits at the 24, 48, and 72 hour observation periods.

According to ISO 10993:10 test criteria, if the difference between the average scores for the extract of the test article and the vehicle control is less than or equal to 1.0, the test article is considered non-irritating. The differences in the mean test and control scores of the test fibers extract dermal observations were less than 1.0 as shown in Table 10, indicating that the requirements of the ISO Intracutaneous Reactivity Test have been met by the test articles.

TABLE 10

| | 0.9% Normal Saline (SN) | | | | | | Cottonseed oil (CSO) | | | | | |
|---|---|---|---|---|---|---|---|---|---|---|---|---|
| | CH | | CHA | | CHAE10 | | CH | | CHA | | CHAE10 | |
| | Test | | Test | | Test | | Test | | Test | | Test | |
| | Fiber | Control | Fiber | Control | Fiber | Control | Fiber | Control | Fiber | Control | Fiber | Control |
| Rabbit 1 | 0.3 | 0 | 0 | 0 | 0 | 0.2 | 1 | 1 | 1.2 | 1 | 2 | 2 |
| Rabbit 2 | 0 | 0 | 0 | 0 | 0 | 0 | 1 | 1 | 1 | 1 | 1 | 1 |
| Rabbit 3 | 0.1 | 0 | 0.2 | 0 | 0 | 0 | 1 | 1 | 1.1 | 1.7 | 1.9 | 2 |
| Avg | 0.4 | 0 | 0.1 | 0 | 0 | 0.1 | 1 | 1 | 1.1 | 1.2 | 1.6 | 1.7 |

| | | | | | | |
|---|---|---|---|---|---|---|
| Results^{a} | 0.4 | 0.1 | 0* | 0 | 0* | 0* |

^{a}comparative results = average test-average control;
*Negative values reported as 0.

Patch Formation

Patches with chitin fiber, chitinous-alginate fiber, chitinous-alginate-vitamin E fiber were formed. Referring to FIG. 9, an example wound dressing 110 is shown to have fiber mat 116 positioned at the center of backing material 114, and covered with a covering 112.

Efficacy Tests

The purpose of this study was to evaluate wound healing response after a topical application of novel wound treatment products in rats at seven and fourteen days post-wound formation. Wound healing studies (rat model, histopathologic evaluation) were performed to evaluate wound healing response after an application of one of the patches as compared to reported wound healing results under similar test conditions using commercially available OPSITE dressing, marketed by Smith and Nephew. A rat wound healing model was used to compare wound healing response after topical application of a novel wound treatment product to that of a control article and the group and methodology applied are listed in Table 11. The fiber CH was chosen as control since it constitutes the backbone of the other two test fibers (CHA and CHAE10). Two wounds were created in each of 30 Sprague Dawley rats. Test or control articles were placed on wounds post-creation. Photographs and wound measurements were obtained post-wound creation, on Days 0, 3, 7, 10, and 14. The wound sites and surrounding tissue were processed by standard histological techniques. Histological analysis and wound measurements were used for comparison.

TABLE 11

| | Number of | | Evaluation Times | Termi- |
|---|---|---|---|---|
| | Animals per | | (Days Post- | nation |
| | Time point | Wound | Wound | Time |
| Group | (Total) | Application | Creation) | (Days) |
| 1 | 5 (10) | Test Article 1 | 3, 7, 10, and 14 | 7 and 14 |
| | | (CHA) | | |
| 2 | 5 (10) | Test Article 2 | 3, 7, 10, and 14 | 7 and 14 |
| | | (CHAE10) | | |
| 3 | 5 (10) | Control Article | 3, 7, 10, and 14 | 7 and 14 |
| | | (CH) | | |

Wound Closure Measurements

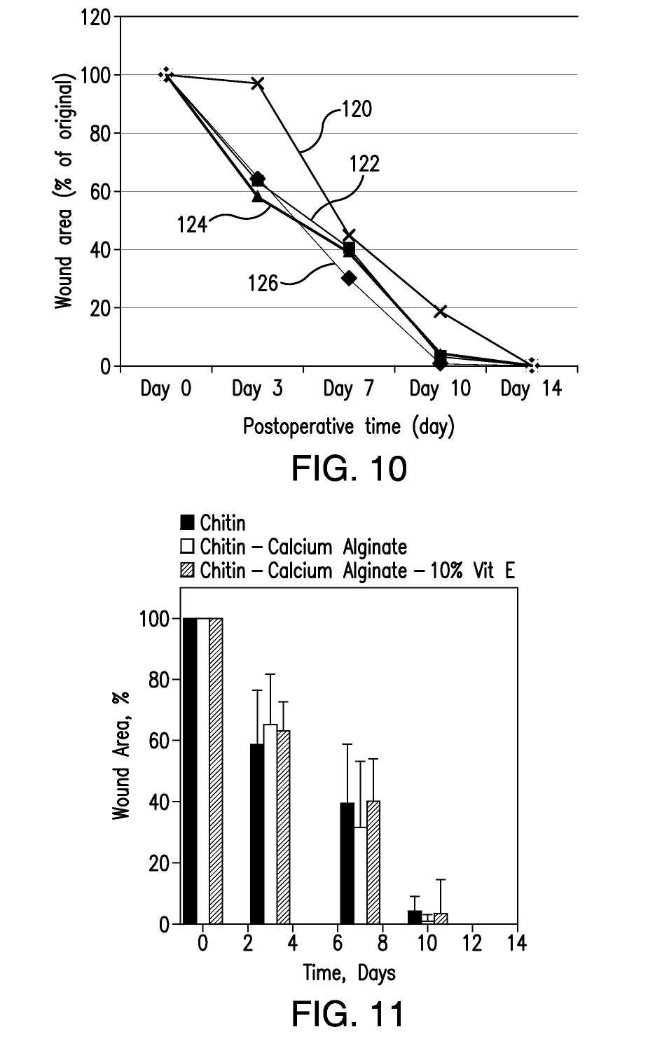

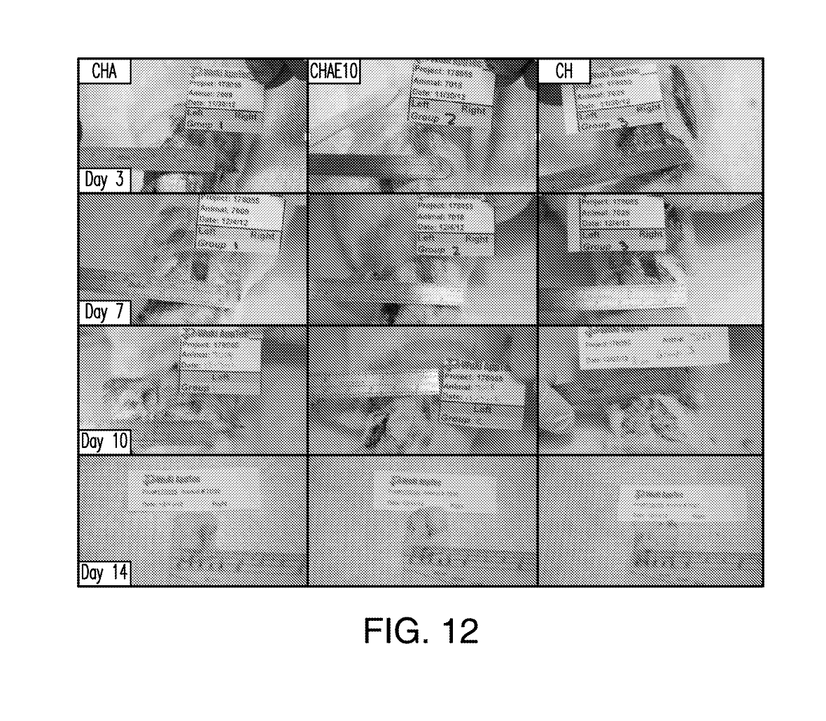

All the tested dressings could be removed from the wound area without causing any further trauma to the animal. Wound healing profile of each test fiber (CHA and CHAE10) was compared to that seen with the control fiber (CH) through wound measurements collected on Days 0, 3, 7, 10 and 14 and the results are listed in Table 12 and plotted in FIGS. 10 and 11, summarizing the wound closure results, expressed as percentage of wound area (average area (per group) of wound measured at Days 3, 7, 10, and 14 post wounding divided by the initial wound area measured on Day 0. As shown in FIG. 10, all the test articles: wound sites treated with chitin patch 124, chitinous-alginate patch 126, chitinous-alginate vitamin E patch 122, and OPSITE patch 120 achieved 95-99% closure by day 10 with complete wound closure (100%) by day 14, indicating that wound sites were healing fast, with chitinous-alginate and chitinous-alginate-vitamin E patches performing better than other tested patches. Mature epidermal architecture with re-epithelialization of normal thickness consistent with normal healing of a full thickness dermal wound were observed in all studies. The chitinous-alginate and chitinous-alginate-vitamin E patches performed better than other products used for wound care, displaying accelerated wound healing properties.

TABLE 12

| | Measurement | | | | | | | | | | | |
|---|---|---|---|---|---|---|---|---|---|---|---|---|
| | Day 3 | | Day 7 | | Day 10 | | Day 14 | | Wound Closure % | | | |
| | N = 20 | Std. | N = 20 | Std. | N = 20 | Std. | N = 20 | Std. | Day | Day | Day | Day |
| Fibers | Mean | Dev. | Mean | Dev. | Mean | Dev. | Mean | Dev. | 3 | 7 | 10 | 14 |
| CH | 42.1 | 9.7 | 19.7 | 11.7 | 0.6 | 1.2 | 0 | 0 | 35.5 | 69.6 | 99.1 | 100 |
| CHA | 41.2 | 7.9 | 26.2 | 9.2 | 2.2 | 6.5 | 0 | 0 | 36.7 | 59.7 | 96.7 | 100 |
| CHAE10 | 37.3 | 10.5 | 25.2 | 12.3 | 2.7 | 2.8 | 0 | 0 | 41.8 | 60.6 | 95.8 | 100 |

Histopathological Evaluation

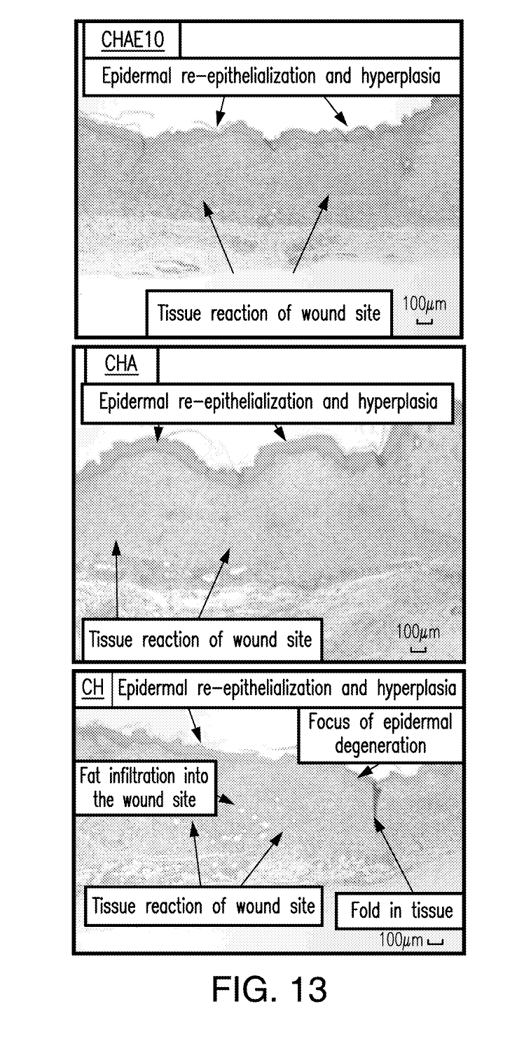

Overall, the tissue response found in the day 7 (Tables 12, 14, 15) and day 14 (Tables 16, 17, 18) at the wound sites treated with test fibers CHA, CHAE10 and control fiber CH is consistent with normal healing of a full thickness dermal wound. On the 7 day wound sites in all three groups (test fibers CHA, CHAE10 and control CH) were comprised of early healing (fibrosis with neovascularization, inflammation and epidermal proliferation) and the 14 day wound sites in all three groups consisted of maturing dermal wounds with differentiation of the fibrosis into dermal collagen fibers with a decrease in the amount of neovascularization and inflammation. On day 7, the repair process was still ongoing, with the epidermal tissue completely covering the dermal wounds dressed with the test fibers CHA and CHAE10, whereas the epidermal tissue was only partially covering the wound dressed with the control fiber CH as illustrated in FIG. 13, showing hematoxylin and eosin (H&E) stained tissue sections of the wound sites at 40× magnification. On day 14, there was more contraction of the wound dressed with the control fiber CH than in the wound sites dressed with the test fibers CHA and CHAE10.

All the fibers displayed accelerated wound healing properties. When compared with published data for the commercially available wound dressing OPSITE™, Wang et al. “Chitosan-Alginate PEC Membrane as a Wound Dressing: Assessment of Incisional Wound Healing”. J. Biomed. Mater. Res. 2002, 63, 601-618, the wound sites dressed with all of the tested fibers stabilized and healed faster. The wound model studies were performed using only one dressing/per animal/per wound site during the entire duration of the study (14 days). For comparison, OPSITE™ patch reported in Wang were reapplied on days 4, 7, and 11, using saline to clean wound before new patch was applied. The chitinous-alginate and chitinous-alginate-vitamin E patches therefore demonstrated significantly superior performance to commercially available dressings.

TABLE 13
Histopathology of 7 day chitin (CH) treated wound sites

| | Animal Number | | | | | | | | | | |
|---|---|---|---|---|---|---|---|---|---|---|---|
| | 7021 | | 7022 | | 7023 | | 7025 | | 7031 | | Average of |
| | Group Number 3 | | | | | | | | | | Wound |
| Wound Site | LT | RT | LT | RT | LT | RT | LT | RT | LT | RT | Sites |

Inflammation (I)

| | | | | | | | | | | | |
|---|---|---|---|---|---|---|---|---|---|---|---|
| Polymorphonuclear | 1 | 1 | 1 | 1 | 1 | 1 | 0 | 1 | 1 | 1 | 1 |
| Cells | | | | | | | | | | | |
| Lymphocytes | 1 | 1 | 1 | 1 | 1 | 1 | 1 | 1 | 2 | 2 | 1 |
| Eosinophils | 0 | 0 | 0 | 0 | 0 | 0 | 0 | 0 | 0 | 0 | 0 |
| Mast Cells | 1 | 1 | 1 | 1 | 1 | 1 | 1 | 1 | 1 | 1 | 1 |
| Plasma Cells | 0 | 0 | 0 | 0 | 0 | 0 | 0 | 0 | 0 | 0 | 0 |
| Macrophages | 3 | 4 | 3 | 3 | 3 | 3 | 3 | 3 | 3 | 4 | 3 |
| Multinucleated Giant Cells | 1 | 1 | 1 | 1 | 1 | 1 | 1 | 1 | 1 | 1 | 1 |
| SUBTOTAL (I) | 7 | 8 | 7 | 7 | 7 | 7 | 6 | 7 | 8 | 9 | 7 |

Regenerative Tissue Response (RTR)

| | | | | | | | | | | | |
|---|---|---|---|---|---|---|---|---|---|---|---|
| Neovascularization | 3 | 4 | 2 | 2 | 3 | 3 | 3 | 3 | 3 | 3 | 3 |
| Fibrous Connective | 3 | 3 | 1 | 1 | 2 | 2 | 3 | 3 | 3 | 3 | 2 |
| Tissue | | | | | | | | | | | |
| Blood Clot | 0 | 0 | 0 | 0 | 0 | 0 | 1 | 0 | 0 | 0 | 0 |
| Epidermal Scab | 1 | 1 | 0 | 0 | 1 | 1 | 1 | 0 | 1 | 1 | 1 |
| Re-epithelialization of | 2 | 2 | 4 | 4 | 4 | 3 | 4 | 4 | 4 | 3 | 3 |
| Wound (Migration) | | | | | | | | | | | |
| Epidermal Differentiation | 2 | 2 | 4 | 4 | 2 | 3 | 4 | 4 | 4 | 2 | 3 |
| in Re-epithelialization | | | | | | | | | | | |
| Hyperplastic Epidermis | 2 | 2 | 4 | 4 | 2 | 3 | 4 | 4 | 4 | 2 | 3 |
| Dermal Collagen | 1 | 1 | 3 | 3 | 2 | 2 | 1 | 1 | 1 | 1 | 2 |
| Remodeling | | | | | | | | | | | |
| SUBTOTAL (RTR) | 14 | 15 | 18 | 18 | 16 | 17 | 21 | 19 | 20 | 15 | 17 |
| TOTAL (I + RTR) | 21 | 23 | 25 | 25 | 23 | 24 | 27 | 26 | 28 | 24 | 25 |

Degenerative Tissue Response (DTR)

| | | | | | | | | | | | |
|---|---|---|---|---|---|---|---|---|---|---|---|
| Epidermal Degeneration | 1 | 1 | 2 | 0 | 2 | 0 | 1 | 1 | 1 | 2 | 1 |
| Abscesses | 0 | 0 | 0 | 0 | 0 | 0 | 0 | 0 | 0 | 0 | 0 |
| Subacute/Chronic | 1 | 0 | 1 | 1 | 1 | 1 | 1 | 1 | 1 | 1 | 1 |
| Hemorrhage | | | | | | | | | | | |
| Necrosis/Apoptosis | 0 | 0 | 0 | 0 | 0 | 0 | 0 | 0 | 0 | 0 | 0 |
| Ulcer | 2 | 3 | 0 | 0 | 0 | 2 | 0 | 0 | 0 | 1 | 1 |
| Granuloma | 0 | 0 | 0 | 0 | 0 | 0 | 0 | 0 | 0 | 0 | 0 |
| SUBTOTAL (DTR) | 4 | 4 | 3 | 1 | 3 | 3 | 2 | 2 | 2 | 4 | 3 |
| (I + RTR) − DTR | 17 | 19 | 22 | 24 | 20 | 21 | 25 | 24 | 26 | 20 | 22 |

Other Tissue Changes

| | | | | | | | | | | | |
|---|---|---|---|---|---|---|---|---|---|---|---|
| Myofiber | 1 | 0 | 0 | 1 | 0 | 0 | 1 | 0 | 1 | 1 | 1 |
| Degeneration/Necrosis | | | | | | | | | | | |
| Myofiber Regeneration | 1 | 1 | 1 | 1 | 1 | 1 | 1 | 1 | 1 | 1 | 1 |
| Fat Infiltration | 1 | 0 | 1 | 1 | 1 | 1 | 0 | 1 | 1 | 1 | 1 |
| Soft Tissue Mineralization | 0 | 0 | 0 | 0 | 0 | 0 | 0 | 0 | 0 | 0 | 0 |
| Hair fibers in Wound | 1 | 0 | 1 | 1 | 0 | 1 | 1 | 1 | 1 | 1 | 1 |
| Dermal Wound Width | 3.57 | 3.60 | 1.86 | 3.86 | 5.18 | 4.23 | 3.82 | 4.30 | 3.54 | 4.12 | 3.81 |
| (millimeters) | | | | | | | | | | | |
| Width of Epidermal Gap | 1.13 | 2.32 | 0 | 0 | 0 | 1.37 | 0 | 0 | 0 | 0.25 | 0.51 |
| (microns) | | | | | | | | | | | |

TABLE 14
Histopathology of 7 day chitin-calcium alginate (CHA) treated wound sites

| | Animal Number | | | | | | | | | | |
|---|---|---|---|---|---|---|---|---|---|---|---|
| | 7001 | | 7002 | | 7003 | | 7004 | | 7005 | | Average of |
| | Group Number 1 | | | | | | | | | | Wound |
| Wound Site | LT | RT | LT | RT | LT | RT | LT | RT | LT | RT | Sites |

Inflammation (I)

| | | | | | | | | | | | |
|---|---|---|---|---|---|---|---|---|---|---|---|
| Polymorphonuclear | 0 | 1 | 1 | 1 | 1 | 1 | 1 | 1 | 0 | 0 | 1 |
| Cells | | | | | | | | | | | |
| Lymphocytes | 1 | 1 | 1 | 1 | 2 | 1 | 1 | 2 | 1 | 1 | 1 |
| Eosinophils | 0 | 0 | 0 | 0 | 0 | 0 | 0 | 0 | 0 | 0 | 0 |
| Mast Cells | 1 | 1 | 1 | 1 | 1 | 1 | 1 | 1 | 1 | 1 | 1 |
| Plasma Cells | 0 | 1 | 1 | 0 | 0 | 0 | 0 | 0 | 0 | 0 | 0 |
| Macrophages | 3 | 3 | 3 | 4 | 3 | 4 | 3 | 4 | 3 | 3 | 3 |
| Multinucleated Giant Cells | 0 | 1 | 1 | 1 | 1 | 1 | 1 | 1 | 1 | 1 | 1 |
| SUBTOTAL (I) | 5 | 8 | 8 | 8 | 8 | 8 | 7 | 9 | 6 | 6 | 7 |

Regenerative Tissue Response (RTR)

| | | | | | | | | | | | |
|---|---|---|---|---|---|---|---|---|---|---|---|
| Neovascularization | 3 | 3 | 3 | 3 | 3 | 4 | 3 | 4 | 3 | 3 | 3 |
| Fibrous Connective | 2 | 3 | 2 | 3 | 2 | 4 | 3 | 3 | 2 | 1 | 3 |
| Tissue | | | | | | | | | | | |
| Blood Clot | 0 | 0 | 0 | 0 | 0 | 0 | 0 | 0 | 0 | 0 | 0 |
| Epidermal Scab | 0 | 2 | 1 | 1 | 0 | 1 | 0 | 1 | 1 | 3 | 1 |
| Re-epithelialization of | 4 | 2 | 4 | 4 | 4 | 3 | 4 | 4 | 4 | 4 | 4 |
| Wound (Migration) | | | | | | | | | | | |
| Epidermal | 4 | 2 | 4 | 4 | 4 | 2 | 4 | 4 | 4 | 4 | 4 |
| Differentiation in Re- | | | | | | | | | | | |
| epithelialization | | | | | | | | | | | |
| Hyperplastic Epidermis | 4 | 2 | 4 | 4 | 4 | 2 | 4 | 3 | 4 | 4 | 4 |
| Dermal Collagen | 2 | 1 | 2 | 1 | 2 | 1 | 1 | 1 | 3 | 3 | 2 |
| Remodeling | | | | | | | | | | | |
| SUBTOTAL (RTR) | 19 | 15 | 20 | 20 | 19 | 17 | 19 | 20 | 21 | 22 | 19 |
| TOTAL (I + RTR) | 24 | 23 | 28 | 28 | 27 | 25 | 26 | 29 | 27 | 28 | 27 |

Degenerative Tissue Response (DTR)

| | | | | | | | | | | | |
|---|---|---|---|---|---|---|---|---|---|---|---|
| Epidermal Degeneration | 0 | 2 | 0 | 2 | 0 | 1 | 0 | 2 | 0 | 0 | 1 |
| Abscesses | 0 | 0 | 0 | 1 | 0 | 0 | 0 | 0 | 0 | 0 | 0 |
| Subacute/Chronic | 1 | 1 | 1 | 1 | 1 | 0 | 1 | 1 | 1 | 1 | 1 |
| Hemorrhage | | | | | | | | | | | |
| Necrosis/Apoptosis | 0 | 0 | 0 | 0 | 0 | 1 | 0 | 0 | 0 | 0 | 0 |
| Ulcer | 0 | 2 | 0 | 0 | 0 | 1 | 0 | 1 | 0 | 0 | 0 |
| Granuloma | 0 | 0 | 0 | 0 | 0 | 1 | 0 | 0 | 0 | 0 | 0 |
| SUBTOTAL (DTR) | 1 | 5 | 1 | 4 | 1 | 4 | 1 | 4 | 1 | 1 | 2 |
| (I + RTR) − DTR | 23 | 18 | 27 | 24 | 26 | 21 | 25 | 25 | 26 | 27 | 24 |

Other Tissue Changes

| | | | | | | | | | | | |
|---|---|---|---|---|---|---|---|---|---|---|---|
| Myofiber | 0 | 0 | 1 | 1 | 0 | 1 | 1 | 1 | 1 | 0 | 1 |
| Degeneration/Necrosis | | | | | | | | | | | |
| Myofiber Regeneration | 1 | 1 | 1 | 1 | 1 | 1 | 1 | 1 | 1 | 1 | 1 |
| Fat Infiltration | 1 | 1 | 0 | 1 | 1 | 0 | 0 | 1 | 0 | 1 | 1 |
| Soft Tissue | 0 | 0 | 0 | 0 | 0 | 0 | 0 | 0 | 0 | 0 | 0 |
| Mineralization | | | | | | | | | | | |
| Hair fibers in Wound | 1 | 1 | 1 | 1 | 1 | 1 | 1 | 1 | 1 | 1 | 1 |
| Dermal Wound Width | 3.49 | 2.95 | 3.78 | 3.46 | 3.35 | 3.15 | 3.64 | 3.91 | 2.37 | 2.14 | 3.22 |
| (millimeters) | | | | | | | | | | | |
| Width of Epidermal Gap | 0 | 0.96 | 0 | 0 | 0 | 0.75 | 0 | 0 | 0 | 0 | 0.17 |
| (microns) | | | | | | | | | | | |

TABLE 15
Histopathology of 7 day chitin-calcium alginate 10% vitamin E (CHAE10) treated wound sites

| | Animal Number | | | | | | | | | | |
|---|---|---|---|---|---|---|---|---|---|---|---|
| | 7011 | | 7012 | | 7013 | | 7014 | | 7015 | | Average of |
| | Group Number . | | | | | | | | | | Wound |
| Wound Site | LT | RT | LT | RT | LT | RT | LT | RT | LT | RT | Sites |

Inflammation (I)

| | | | | | | | | | | | |
|---|---|---|---|---|---|---|---|---|---|---|---|
| Polymorphonuclear | 1 | 1 | 1 | 1 | 1 | 1 | 1 | 1 | 0 | 0 | 1 |
| Cells | | | | | | | | | | | |
| Lymphocytes | 2 | 2 | 1 | 1 | 2 | 1 | 2 | 1 | 1 | 1 | 1 |
| Eosinophils | 0 | 0 | 0 | 0 | 0 | 0 | 0 | 0 | 0 | 0 | 0 |
| Mast Cells | 1 | 1 | 1 | 1 | 1 | 1 | 1 | 1 | 1 | 1 | 1 |
| Plasma Cells | 0 | 0 | 0 | 0 | 0 | 0 | 1 | 0 | 0 | 0 | 0 |
| Macrophages | 3 | 3 | 3 | 4 | 2 | 3 | 3 | 3 | 3 | 1 | 3 |
| Multinucleated Giant Cells | 1 | 1 | 1 | 1 | 1 | 1 | 1 | 1 | 1 | 1 | 1 |
| SUBTOTAL (I) | 8 | 8 | 7 | 8 | 7 | 7 | 9 | 7 | 6 | 4 | 7 |

Regenerative Tissue Response (RTR)

| | | | | | | | | | | | |
|---|---|---|---|---|---|---|---|---|---|---|---|
| Neovascularization | 3 | 3 | 3 | 3 | 3 | 3 | 3 | 3 | 3 | 3 | 3 |
| Fibrous Connective | 2 | 2 | 2 | 3 | 2 | 3 | 3 | 1 | 2 | 1 | 2 |
| Tissue | | | | | | | | | | | |
| Blood Clot | 0 | 0 | 0 | 0 | 0 | 0 | 0 | 0 | 0 | 0 | 0 |
| Epidermal Scab | 0 | 1 | 1 | 2 | 1 | 0 | 0 | 0 | 1 | 0 | 1 |
| Re-epithelialization of | 4 | 4 | 3 | 2 | 4 | 4 | 4 | 4 | 4 | 4 | 4 |
| Wound (Migration) | | | | | | | | | | | |
| Epidermal | 4 | 4 | 2 | 2 | 4 | 4 | 4 | 4 | 4 | 4 | 4 |
| Differentiation in Re- | | | | | | | | | | | |
| epithelialization | | | | | | | | | | | |
| Hyperplastic Epidermis | 4 | 4 | 2 | 2 | 4 | 4 | 4 | 4 | 4 | 4 | 4 |
| Dermal Collagen | 2 | 1 | 2 | 1 | 3 | 1 | 1 | 3 | 2 | 4 | 2 |
| Remodeling | | | | | | | | | | | |
| SUBTOTAL (RTR) | 19 | 19 | 15 | 15 | 21 | 19 | 19 | 19 | 20 | 20 | 19 |
| TOTAL (I + RTR) | 27 | 27 | 22 | 23 | 28 | 26 | 28 | 26 | 26 | 24 | 26 |

Degenerative Tissue Response (DTR)

| | | | | | | | | | | | |
|---|---|---|---|---|---|---|---|---|---|---|---|
| Epidermal Degeneration | 0 | 1 | 2 | 0 | 1 | 1 | 0 | 0 | 1 | 1 | 1 |
| Abscesses | 0 | 0 | 0 | 0 | 0 | 0 | 0 | 0 | 0 | 0 | 0 |
| Subacute/Chronic | 1 | 0 | 1 | 0 | 1 | 0 | 1 | 1 | 1 | 1 | 1 |
| Hemorrhage | | | | | | | | | | | |
| Necrosis/Apoptosis | 0 | 0 | 0 | 0 | 0 | 0 | 0 | 0 | 0 | 0 | 0 |
| Ulcer | 0 | 0 | 1 | 3 | 0 | 0 | 0 | 0 | 0 | 0 | 0 |
| Granuloma | 0 | 0 | 0 | 1 | 0 | 0 | 0 | 0 | 0 | 0 | 0 |
| SUBTOTAL (DTR) | 1 | 1 | 4 | 4 | 2 | 1 | 1 | 1 | 2 | 2 | 2 |
| (I + RTR) − DTR | 26 | 26 | 18 | 19 | 26 | 25 | 27 | 25 | 24 | 22 | 24 |

Other Tissue Changes

| | | | | | | | | | | | |
|---|---|---|---|---|---|---|---|---|---|---|---|
| Myofiber | 0 | 0 | 1 | 1 | 0 | 1 | 0 | 1 | 0 | 1 | 1 |
| Degeneration/Necrosis | | | | | | | | | | | |
| Myofiber Regeneration | 1 | 1 | 1 | 1 | 1 | 1 | 1 | 1 | 1 | 1 | 1 |
| Fat Infiltration | 1 | 0 | 0 | 1 | 1 | 0 | 1 | 1 | 0 | 2 | 1 |
| Soft Tissue | 0 | 0 | 0 | 0 | 0 | 0 | 0 | 0 | 0 | 0 | 0 |
| Mineralization | | | | | | | | | | | |
| Hair fibers in Wound | 1 | 1 | 1 | 0 | 1 | 1 | 1 | 1 | 0 | 1 | 1 |
| Dermal Wound Width | 3.97 | 4.68 | 4.47 | 3.52 | 3.99 | 3.17 | 3.47 | 4.30 | 3.92 | 4.13 | 3.96 |
| (millimeters) | | | | | | | | | | | |
| Width of Epidermal Gap | 0 | 0 | 0.45 | 1.62 | 0 | 0 | 0 | 0 | 0 | 0 | 0.21 |
| (microns) | | | | | | | | | | | |

TABLE 16
Histopathology of 14 day chitin (CH) treated wound sites

| | Animal Number | | | | | | | | | | |
|---|---|---|---|---|---|---|---|---|---|---|---|
| | 7026 | | 7027 | | 7028 | | 7029 | | 7030 | | Average of |
| | Group Number 3 | | | | | | | | | | Wound |
| Wound Site | LT | RT | LT | RT | LT | RT | LT | RT | LT | RT | Sites |

Inflammation (I)

| | | | | | | | | | | | |
|---|---|---|---|---|---|---|---|---|---|---|---|
| Polymorphonuclear | 1 | 0 | 0 | 1 | 1 | 0 | 0 | 0 | 1 | 1 | 1 |
| Cells | | | | | | | | | | | |
| Lymphocytes | 1 | 1 | 1 | 1 | 1 | 1 | 1 | 1 | 1 | 1 | 1 |
| Eosinophils | 0 | 0 | 0 | 0 | 0 | 0 | 0 | 0 | 0 | 0 | 0 |
| Mast Cells | 1 | 1 | 1 | 1 | 1 | 1 | 1 | 1 | 1 | 1 | 1 |
| Plasma Cells | 0 | 1 | 0 | 1 | 0 | 0 | 0 | 0 | 0 | 0 | 0 |
| Macrophages | 1 | 1 | 1 | 1 | 1 | 1 | 2 | 1 | 3 | 1 | 1 |
| Multinucleated Giant Cells | 0 | 1 | 1 | 1 | 0 | 0 | 1 | 1 | 1 | 0 | 1 |
| SUBTOTAL (I) | 4 | 5 | 4 | 6 | 4 | 3 | 5 | 4 | 7 | 4 | 5 |

Regenerative Tissue Response (RTR)

| | | | | | | | | | | | |
|---|---|---|---|---|---|---|---|---|---|---|---|
| Neovascularization | 2 | 2 | 2 | 2 | 2 | 2 | 2 | 2 | 2 | 2 | 2 |
| Fibrous Connective | 1 | 1 | 1 | 1 | 1 | 1 | 1 | 1 | 1 | 1 | 1 |
| Tissue | | | | | | | | | | | |
| Blood Clot | 0 | 0 | 0 | 0 | 0 | 0 | 0 | 0 | 0 | 0 | 0 |
| Epidermal Scab | 1 | 0 | 0 | 0 | 0 | 1 | 0 | 0 | 1 | 0 | 0 |
| Re-epithelialization of | 4 | 4 | 4 | 4 | 4 | 4 | 4 | 4 | 4 | 4 | 4 |
| Wound (Migration) | | | | | | | | | | | |
| Epidermal | 4 | 4 | 4 | 4 | 4 | 4 | 4 | 4 | 4 | 4 | 4 |
| Differentiation in Re- | | | | | | | | | | | |
| epithelialization | | | | | | | | | | | |
| Hyperplastic Epidermis | 4 | 4 | 4 | 4 | 4 | 4 | 4 | 4 | 4 | 4 | 4 |
| Dermal Collagen | 4 | 4 | 4 | 4 | 4 | 4 | 4 | 4 | 4 | 4 | 4 |
| Remodeling | | | | | | | | | | | |
| SUBTOTAL (RTR) | 20 | 19 | 19 | 19 | 19 | 20 | 19 | 19 | 20 | 19 | 19 |
| TOTAL (I + RTR) | 24 | 24 | 23 | 25 | 23 | 23 | 24 | 23 | 27 | 23 | 24 |

Degenerative Tissue Response (DTR)

| | | | | | | | | | | | |
|---|---|---|---|---|---|---|---|---|---|---|---|
| Epidermal Degeneration | 0 | 0 | 1 | 1 | 0 | 1 | 1 | 1 | 1 | 1 | 1 |
| Abscesses | 0 | 0 | 0 | 0 | 0 | 0 | 0 | 0 | 0 | 0 | 0 |
| Subacute/Chronic | 1 | 1 | 1 | 1 | 1 | 1 | 1 | 1 | 1 | 1 | 1 |
| Hemorrhage | | | | | | | | | | | |
| Necrosis/Apoptosis | 0 | 0 | 0 | 0 | 0 | 0 | 0 | 0 | 0 | 0 | 0 |
| Ulcer | 0 | 0 | 0 | 0 | 0 | 0 | 0 | 0 | 0 | 0 | 0 |
| Granuloma | 0 | 0 | 0 | 0 | 0 | 0 | 0 | 0 | 0 | 0 | 0 |
| SUBTOTAL (DTR) | 1 | 1 | 2 | 2 | 1 | 2 | 2 | 2 | 2 | 2 | 2 |
| (I + RTR) − DTR | 23 | 23 | 21 | 23 | 22 | 21 | 22 | 21 | 25 | 21 | 22 |

Other Tissue Changes

| | | | | | | | | | | | |
|---|---|---|---|---|---|---|---|---|---|---|---|
| Myofiber | 1 | 1 | 1 | 1 | 1 | 1 | 1 | 1 | 1 | 1 | 1 |
| Degeneration/Necrosis | | | | | | | | | | | |
| Myofiber Regeneration | 1 | 1 | 1 | 1 | 1 | 1 | 1 | 1 | 1 | 1 | 1 |
| Fat Infiltration | 0 | 1 | 0 | 0 | 0 | 1 | 1 | 0 | 0 | 0 | 0 |
| Soft Tissue | 0 | 0 | 0 | 0 | 0 | 0 | 0 | 0 | 0 | 0 | 0 |
| Mineralization | | | | | | | | | | | |
| Hair fibers in Wound | 0 | 1 | 1 | 1 | 0 | 0 | 0 | 1 | 1 | 0 | 1 |
| Dermal Wound Width | 1.64 | 0.71 | 2.42 | 1.14 | 1.69 | 0.41 | 1.44 | 1.59 | 2.25 | 1.54 | 1.48 |
| (millimeters) | | | | | | | | | | | |
| Width of Epidermal Gap | 0 | 0 | 0 | 0 | 0 | 0 | 0 | 0 | 0 | 0 | 0 |
| (microns) | | | | | | | | | | | |

TABLE 17
Histopathology of 14 day chitin-calcium alginate (CHA) treated wound sites

| | Animal Number | | | | | | | | | | |
|---|---|---|---|---|---|---|---|---|---|---|---|
| | 7006 | | 7007 | | 7008 | | 7009 | | 7010 | | Average of |
| | Group Number 1 | | | | | | | | | | Wound |
| Wound Site | LT | RT | LT | RT | LT | RT | LT | RT | LT | RT | Sites |

Inflammation (I)

| | | | | | | | | | | | |
|---|---|---|---|---|---|---|---|---|---|---|---|
| Polymorphonuclear | 0 | 0 | 0 | 0 | 0 | 1 | 0 | 0 | 0 | 1 | 0 |
| Cells | | | | | | | | | | | |
| Lymphocytes | 1 | 1 | 1 | 1 | 1 | 1 | 1 | 1 | 1 | 1 | 1 |
| Eosinophils | 0 | 0 | 0 | 0 | 0 | 0 | 0 | 0 | 0 | 0 | 0 |
| Mast Cells | 0 | 1 | 1 | 0 | 1 | 1 | 1 | 1 | 1 | 1 | 1 |
| Plasma Cells | 0 | 0 | 1 | 0 | 0 | 0 | 0 | 0 | 0 | 0 | 0 |
| Macrophages | 1 | 1 | 2 | 1 | 1 | 1 | 1 | 2 | 2 | 1 | 1 |
| Multinucleated Giant Cells | 0 | 1 | 1 | 1 | 1 | 1 | 1 | 1 | 1 | 1 | 1 |
| SUBTOTAL (I) | 2 | 4 | 6 | 3 | 4 | 5 | 4 | 5 | 5 | 5 | 4 |

Regenerative Tissue Response (RTR)

| | | | | | | | | | | | |
|---|---|---|---|---|---|---|---|---|---|---|---|
| Neovascularization | 2 | 2 | 2 | 3 | 2 | 2 | 2 | 2 | 2 | 1 | 2 |
| Fibrous Connective | 1 | 1 | 1 | 1 | 1 | 1 | 1 | 1 | 1 | 1 | 1 |
| Tissue | | | | | | | | | | | |
| Blood Clot | 0 | 0 | 0 | 0 | 0 | 0 | 0 | 0 | 0 | 0 | 0 |
| Epidermal Scab | 0 | 0 | 0 | 0 | 0 | 0 | 0 | 4 | 0 | 0 | 0 |
| Re-epithelialization of | 4 | 4 | 4 | 4 | 4 | 4 | 4 | 4 | 4 | 4 | 4 |
| Wound (Migration) | | | | | | | | | | | |
| Epidermal | 4 | 4 | 4 | 4 | 4 | 4 | 4 | 4 | 4 | 4 | 4 |
| Differentiation in Re- | | | | | | | | | | | |
| epithelialization | | | | | | | | | | | |
| Hyperplastic Epidermis | 4 | 4 | 4 | 4 | 4 | 4 | 4 | 4 | 4 | 4 | 4 |
| Dermal Collagen | 4 | 4 | 4 | 4 | 4 | 4 | 4 | 4 | 4 | 4 | 4 |
| Remodeling | | | | | | | | | | | |
| SUBTOTAL (RTR) | 19 | 19 | 19 | 20 | 19 | 19 | 19 | 23 | 19 | 18 | 19 |
| TOTAL (I + RTR) | 21 | 23 | 25 | 23 | 23 | 24 | 23 | 28 | 24 | 23 | 24 |

Degenerative Tissue Response (DTR)

| | | | | | | | | | | | |
|---|---|---|---|---|---|---|---|---|---|---|---|
| Epidermal Degeneration | 0 | 0 | 2 | 1 | 1 | 0 | 1 | 0 | 0 | 0 | 1 |
| Abscesses | 0 | 0 | 0 | 0 | 0 | 0 | 0 | 0 | 0 | 0 | 0 |
| Subacute/Chronic | 1 | 1 | 1 | 1 | 1 | 1 | 1 | 1 | 1 | 1 | 1 |
| Hemorrhage | | | | | | | | | | | |
| Necrosis/Apoptosis | 0 | 0 | 0 | 0 | 0 | 0 | 0 | 0 | 0 | 0 | 0 |
| Ulcer | 0 | 0 | 0 | 0 | 0 | 0 | 0 | 0 | 0 | 0 | 0 |
| Granuloma | 0 | 0 | 0 | 0 | 0 | 0 | 0 | 0 | 0 | 0 | 0 |
| SUBTOTAL (DTR) | 1 | 1 | 3 | 2 | 2 | 1 | 2 | 1 | 1 | 1 | 2 |
| (I + RTR) − DTR | 20 | 22 | 22 | 21 | 21 | 23 | 21 | 27 | 23 | 22 | 22 |

Other Tissue Changes

| | | | | | | | | | | | |
|---|---|---|---|---|---|---|---|---|---|---|---|
| Myofiber | 0 | 1 | 0 | 1 | 0 | 1 | 0 | 1 | 1 | 1 | 1 |
| Degeneration/Necrosis | | | | | | | | | | | |
| Myofiber Regeneration | 1 | 1 | 1 | 1 | 1 | 1 | 1 | 1 | 1 | 1 | 1 |
| Fat Infiltration | 0 | 0 | 1 | 0 | 0 | 0 | 0 | 1 | 0 | 0 | 0 |
| Soft Tissue | 0 | 0 | 0 | 0 | 0 | 0 | 0 | 0 | 0 | 0 | 0 |
| Mineralization | | | | | | | | | | | |
| Hair fibers in Wound | 0 | 1 | 1 | 1 | 1 | 1 | 1 | 1 | 1 | 1 | 1 |
| Dermal Wound Width | 1.54 | 2.55 | 2.74 | 2.45 | 2.26 | 2.30 | 1.41 | 1.58 | 2.27 | 0.65 | 1.98 |
| (millimeters) | | | | | | | | | | | |
| Width of Epidermal | 0 | 0 | 0 | 0 | 0 | 0 | 0 | 0 | 0 | 0 | 0 |
| Gap (microns) | | | | | | | | | | | |

TABLE 18
Histopathology of 7 day chitin-calcium alginate 10% vitamin E (CHAE10) treated wound sites

| | Animal Number | | | | | | | | | | |
|---|---|---|---|---|---|---|---|---|---|---|---|
| | 7016 | | 7017 | | 7018 | | 7019 | | 7020 | | Average of |
| | Group Number 2 | | | | | | | | | | Wound |
| Wound Site | LT | RT | LT | RT | LT | RT | LT | RT | LT | RT | Sites |

Inflammation (I)

| | | | | | | | | | | | |
|---|---|---|---|---|---|---|---|---|---|---|---|
| Polymorphonuclear | 1 | 1 | 1 | 0 | 0 | 0 | 0 | 0 | 1 | 1 | 1 |
| Cells | | | | | | | | | | | |
| Lymphocytes | 1 | 1 | 1 | 2 | 2 | 1 | 1 | 1 | 1 | 1 | 1 |
| Eosinophils | 0 | 0 | 0 | 0 | 0 | 0 | 0 | 0 | 0 | 0 | 0 |
| Mast Cells | 1 | 1 | 1 | 1 | 1 | 1 | 1 | 1 | 1 | 1 | 1 |
| Plasma Cells | 0 | 0 | 0 | 0 | 0 | 1 | 0 | 1 | 0 | 0 | 0 |
| Macrophages | 1 | 2 | 1 | 2 | 1 | 1 | 1 | 1 | 1 | 2 | 1 |
| Multinucleated Giant Cells | 1 | 1 | 0 | 0 | 1 | 1 | 1 | 1 | 1 | 1 | 1 |
| SUBTOTAL (I) | 5 | 6 | 4 | 5 | 5 | 5 | 4 | 5 | 5 | 6 | 5 |

Regenerative Tissue Response (RTR)

| | | | | | | | | | | | |
|---|---|---|---|---|---|---|---|---|---|---|---|
| Neovascularization | 2 | 2 | 2 | 2 | 2 | 2 | 2 | 2 | 2 | 2 | 2 |
| Fibrous Connective | 1 | 1 | 1 | 1 | 1 | 1 | 1 | 1 | 1 | 1 | 1 |
| Tissue | | | | | | | | | | | |
| Blood Clot | 0 | 0 | 0 | 0 | 0 | 0 | 0 | 0 | 0 | 0 | 0 |
| Epidermal Scab | 0 | 2 | 0 | 0 | 0 | 0 | 0 | 0 | 0 | 0 | 0 |
| Re-epithelialization of | 4 | 4 | 4 | 4 | 4 | 4 | 4 | 4 | 4 | 4 | 4 |
| Wound (Migration) | | | | | | | | | | | |
| Epidermal | 4 | 4 | 4 | 4 | 4 | 4 | 4 | 4 | 4 | 4 | 4 |
| Differentiation in Re- | | | | | | | | | | | |
| epithelialization | | | | | | | | | | | |
| Hyperplastic Epidermis | 4 | 4 | 4 | 4 | 4 | 4 | 4 | 4 | 4 | 4 | 4 |
| Dermal Collagen | 4 | 4 | 4 | 4 | 4 | 4 | 4 | 4 | 4 | 4 | 4 |
| Remodeling | | | | | | | | | | | |
| SUBTOTAL (RTR) | 19 | 19 | 19 | 19 | 19 | 19 | 19 | 19 | 19 | 19 | 19 |
| TOTAL (I + RTR) | 24 | 25 | 23 | 24 | 24 | 24 | 23 | 24 | 24 | 25 | 24 |

Degenerative Tissue Response (DTR)

| | | | | | | | | | | | |
|---|---|---|---|---|---|---|---|---|---|---|---|
| Epidermal Degeneration | 1 | 1 | 1 | 0 | 1 | 1 | 1 | 1 | 0 | 1 | 1 |
| Abscesses | 0 | 0 | 0 | 0 | 0 | 0 | 0 | 0 | 0 | 0 | 0 |
| Subacute/Chronic | 1 | 1 | 1 | 1 | 1 | 1 | 1 | 1 | 1 | 1 | 1 |
| Hemorrhage | | | | | | | | | | | |
| Necrosis/Apoptosis | 0 | 0 | 0 | 0 | 0 | 0 | 0 | 0 | 0 | 0 | 0 |
| Ulcer | 0 | 0 | 0 | 0 | 0 | 0 | 0 | 0 | 0 | 0 | 0 |
| Granuloma | 0 | 0 | 0 | 0 | 0 | 0 | 0 | 0 | 0 | 0 | 0 |
| SUBTOTAL (DTR) | 2 | 2 | 2 | 1 | 2 | 2 | 2 | 2 | 1 | 2 | 2 |
| (I + RTR) − DTR | 22 | 23 | 21 | 23 | 22 | 22 | 21 | 22 | 23 | 23 | 22 |

Other Tissue Changes

| | | | | | | | | | | | |
|---|---|---|---|---|---|---|---|---|---|---|---|
| Myofiber | 0 | 1 | 1 | 1 | 1 | 1 | 1 | 1 | 1 | 1 | 1 |
| Degeneration/Necrosis | | | | | | | | | | | |
| Myofiber Regeneration | 1 | 0 | 1 | 1 | 1 | 1 | 1 | 1 | 1 | 1 | 1 |
| Fat Infiltration | 0 | 0 | 0 | 0 | 0 | 0 | 1 | 0 | 1 | 0 | 0 |
| Soft Tissue | 0 | 0 | 0 | 0 | 0 | 0 | 0 | 0 | 0 | 0 | 0 |
| Mineralization | | | | | | | | | | | |
| Hair fibers in Wound | 1 | 1 | 0 | 0 | 1 | 0 | 1 | 1 | 1 | 1 | 1 |
| Dermal Wound Width | 2.45 | 3.92 | 2.21 | 1.54 | 1.81 | 1.34 | 2.53 | 1.71 | 1.28 | 0.99 | 1.98 |
| (millimeters) | | | | | | | | | | | |
| Width of Epidermal | 0 | 0 | 0 | 0 | 0 | 0 | 0 | 0 | 0 | 0 | 0 |
| Gap (microns) | | | | | | | | | | | |

Since many possible aspects may be made of the invention without departing from the scope thereof, it is to be understood that all matter herein set forth or shown in the accompanying drawings is to be interpreted as illustrative and not in a limiting sense.

1. A composite fiber comprising at least 80% by weight of combined chitinous and alginate components in a predetermined ratio having a continuous and homogenous morphology.

2. The composite fiber of claim 1, wherein the weight ratio between the chitinous component and the alginate component is at least 2:1.

3. The composite fiber of claim 1, wherein the weight ratio between the chitinous component and the alginate component is at least 3:1.

4. The composite fiber of claim 1, wherein the weight ratio between the chitinous component and the alginate component is at least 4:1.

5. The composite fiber of claim 1, wherein the composite fiber further comprising an additive.

6. The composite fiber of claim 1, wherein the additive is vitamins, nutraceuticals, non-steroidal anti-inflammatory drugs, anesthetics, analgesics, or ionic liquid active pharmaceutical ingredients.

7. The composite fiber of claim 1, wherein the additive is vitamin E that is about 10% by weight of the composite fiber and the combined chitin and alginate component is about 90% by weight of the composite fiber.

8. The composite fiber of claim 1, wherein the linear mass density of the composite fiber is at least 10 times of the linear mass density of alginate fibers.

9. The composite fiber of claim 1, wherein the moisture content of the composite fiber is at least 50% less than the moisture content of alginate fibers.

10. The composite fiber of claim 1, wherein the tenacity of the composite fiber is at least 10% more than the tenacity of alginate fibers.

11. The composite fiber of claim 1, wherein the percent of elongation of the composite fiber is at least 30% less than the percent of elongation of alginate fibers.

12. The composite fiber of claim 1, wherein the swelling of the composite fiber in water is comparable to the swelling of alginate fibers.

13. The composite fiber of claim 1, wherein the composite fiber is thermal stable at least up to 150° C.

14. The composite fiber of claim 1, wherein the composite fiber is antimicrobial.

15. The composite fiber of claim 1, wherein the composite fiber promotes wound healing.

16. The composite fiber of claim 1, wherein the composite fiber is biodegradable.

17. The composite fiber of claim 1, wherein the composite fiber is biocompatible.

18. The composite fiber of claim 1, wherein the chitin component of the fiber is directly extracted from chitin biomass and has the molecular weight comparable to the chitin in the chitin biomass.

19. A method of using the composite fiber of claim 1, wherein the fiber is used as thread for stitching wound.

20. A wound dressing comprising the composite fibers of claim 1.

21. The wound dressing of claim 1, further comprising a patch that the composite fibers are attached to.

22. A method of healing a wound with the wound dressing of claim 20, the method comprising applying the wound dressing to the wound with the composite fibers directly contacting the wound to speed wound healing.

23. A method of forming composite fiber of claim 1, the method comprising dissolving chitinous biomass and alginate containing biomass in a predetermined ratio in a ionic liquid to form a chitinous-alginate solution before casting the solution to a coagulation solvent to form the composite fiber having a continuous and homogenous morphology.

24. The method of claim 23, wherein the chitinous-alginate solution further comprising an additive.

25. The method of claim 24, wherein the additive is vitamin E.
